(19) United States
(12) Patent Application Publication
(10) Pub. No.: US 2023/0321209 A1
(43) Pub. Date: Oct. 12, 2023

(54) MODIFIED MYCOBACTERIUM BOVIS VACCINES

(71) Applicant: THE UNIVERSITY OF HELSINKI, Helsinki (FI)

(72) Inventors: Erkko Ylösmäki, Helsinki (FI); Manlio Fusciello, Helsinki (FI); Beatriz Martins, Helsinki (FI); Sara Feola, Helsinki (FI); Jacopo Chiaro, Helsinki (FI); Vincenzo Cerullo, Helsinki (FI)

(73) Assignee: Valo Therapeutics Oy, Helsinki (FI)

(21) Appl. No.: 18/043,923

(22) PCT Filed: Aug. 27, 2021
(86) PCT No.: PCT/EP2021/073744

§ 371 Date: Mar. 3, 2023

(30) Foreign Application Priority Data
Sep. 3, 2020 (GB) 2013824.4
Feb. 17, 2021 (GB) 2102211.6
Jul. 8, 2021 (GB) 2109893.4

(51) Int. Cl. A61K 39/00; A61K 39/04; A61P 35/00; A61K 45/06; A61K 39/39

(52) U.S. Cl. CPC A61K 39/001156; A61K 39/04; A61P 35/00; A61K 39/0011; A61K 45/06; A61K 39/39; A61K 2039/522; A61K 2039/70; A61K 2039/627; A61K 2039/6006; A61K 2039/55516; A61K 2039/5254

(57) ABSTRACT

The invention concerns a modified bacteria; a pharmaceutical composition comprising same; and a method of preventing or treating disease particularly, but not exclusively, cancer or an infectious disease using same.

### CROSS REFERENCE TO RELATED APPLICATIONS

This is the U.S. National Stage of International Application No. PCT/EP2021/073744, filed Aug. 27, 2021, which was published in English under PCT Article 21(2), which in turn claims the benefit of Great Britain Application No. 2013824.4, filed Sep. 3, 2020, Great Britain Application No. 2102211.6, filed Feb. 17, 2021, and Great Britain Application No. 2109893.4, filed Jul. 8, 2021. PCT/EP2021/073744 is herein incorporated by reference in its entirety.

### FIELD OF THE INVENTION

The invention concerns a modified bacteria; a pharmaceutical composition comprising same; and a method of preventing or treating disease particularly, but not exclusively, cancer or an infectious disease using same.

### INCORPORATION OF ELECTRONIC SEQUENCE LISTING

The electronic sequence listing, submitted herewith as a txt file named sequence listing.txt (21,006 bytes), created on Feb. 9, 2023, is herein incorporated by reference in its entirety.

### BACKGROUND OF THE INVENTION

The use of modified bacteria as agents to prevent or treat disease is known, particularly in relation to vaccine therapy where, typically, an attenuated form of the bacteria is administered to stimulate the immune system and so elicit a defence against subsequent infection with the wild-type bacteria. Notably, but not exclusively, such vaccines have been developed against diseases caused by infection with the following bacteria: Haemophilus influenzae, Streptococcus pneumoniae, Bordetella pertussis, Vibrio cholerae, Clostridium tetani, Mycobacteriumn tuberculosis, Salmonella typhi, Bacillus anthracis.

Bacterial vaccines can be classified into different types—that is, toxoids, subunit vaccines, killed whole cell vaccines, and live attenuated vaccines. Live bacterial vaccines have the advantage that they can express multiple antigens, can be mass produced and can induce a strong immune response.

Invasive bacteria such as Salmonella, Listeria, Yersinia, Shigella and Mycobacterium bovis (Bacillus Calmette-Guérin or BCG) have been used as vaccines or vaccine vectors, capable of mounting potent humoral and cellular immune responses. Since these are pathogenic bacteria they are attenuated to generate suitable non-pathogenic vaccine strains. Many attenuated strains have been reported that are non-pathogenic and have limited proliferative capacity in vivo.

However, uncertainties regarding the efficacy and mechanisms of action of BCG vaccines remain. Although the protective effect of BCG against disseminated Tuberculosis in young children is reasonably well established, BCG vaccination in adults has demonstrated variable efficacy in clinical trials, and there is no convincing evidence for its protective effect in HIV-infected individuals, a population at high risk of TB disease progression following infection.

BCG is prepared from a strain of the attenuated (virulence-reduced) live bovine tuberculosis bacillus, Mycobacterium bovis, that has lost its ability to cause disease in humans. Because the living bacilli evolve to make the best use of available nutrients, they become less well-adapted to human blood and typically can no longer induce disease when introduced into a human host. However, they are similar enough to their wild-type ancestors to provide some degree of immunity against human tuberculosis. The BCG vaccine can be anywhere from 0 to 80% effective in preventing tuberculosis for a duration of 15 years; however, its protective effect appears to vary according to geography and the lab in which the vaccine strain was grown.

A number of different companies make BCG vaccine, sometimes using different genetic substrains of the bacterium: OncoTice using the substrain TICE, developed by Organon Laboratories (Merck & Co.), Pacis BCG (Dianon Systems), Evans Vaccines (PowderJect Pharmaceuticals), BCG (Statens Serum Institut in Denmark) BCG (Japan BCG Laboratory).

Whilst there is currently a drive to manufacture a viral anti-cancer vaccine, to date, much of the work has involved the use of modified oncolytic viruses.

At the beginning of the century, oncolytic viruses were perceived as active agents in cancer treatment, acting solely through their inherent ability to lyse tumor cells, via oncolysis. More recently, they have been investigated because of their ability to release tumor antigens from cancer cells (upon oncolysis) for activating the immune system.

In this context it is known that BCG is an intracellular pathogen that can modulate the tumour microenvironment (TME) by multiple mechanisms including an induction of a massive secretion of chemokines and cytokines that recruit T cells and other immune cells to the TME, as well as by polarization of M2 macrophages towards a more M1-like phenotype. Recently it was shown that BCG treatment led to enhanced activation and reduced exhaustion of tumour-specific T cells, leading to enhanced effector functions and that BCG-induced bladder cancer elimination required tumour-specific CD4+ and CD8+ T cells, but not T cells specific for BCG antigens.

The current invention concerns a novel agent for preventing and/or treating disease. The invention can be used in the prevention and/or treatment of an infectious disease such as any of those listed above and particularly including a respiratory infection, for example, TB, influenza, SARS, MERS, other coronavirus diseases (such as COVID-19) or the common cold. Further, the invention can be used in the prevention and/or treatment of non-infectious diseases such as cancer or autoimmune diseases.

Statements of the Invention

According to a first aspect of the invention there is provided a live attenuated Mycobacterium bovis (BCG) for use in humans to prevent or treat a disease wherein said BCG is coated with a plurality of peptide antigens capable of eliciting an immune reaction active against said disease in said humans and wherein said peptides are attached to said bacteria using a poly-lysine or poly-arginine peptide linker.

We have termed this modified BCG in accordance with the invention PeptiBAC (peptide-coated bacillus Calmette-Guérin).

In a preferred embodiment of the invention said peptide antigen is an antigen that is associated with said disease and so can be used to elicit an immune response whereby one is protected from said disease or, at least, said disease is less severe than it would otherwise be.

More specifically when treating an infection, particularly a respiratory infection such as a corona virus infection (e.g. SARS-CoV-2), the PeptiBAC platform uses infection associated antigens (viral antigens), preferably such as the ones derived from SARS-CoV-2 which are endogenously expressed by the pathogen. Various viral MHC class I and/or II epitopes deriving from e.g. VME1, AP3A, R1AB, R1A, NS7B, NCAP and Spike proteins can be used to coat the modified BCG. Ideally the PeptiBAC is administered intradermally or intranasally when treating a respiratory infection.

In a preferred embodiment of the invention said disease is an infection and said peptide antigen comprises at least one of the following peptides, ideally attached covalently or non-covalently onto the bacterial envelope without having been genetically encoded by said BCG bacterial vector:

i)
[SEQ ID NO: 1]
IAMACLVGLMWLSYFIASFRLFAR derived from VME1;
ii)[SEQ ID NO: 2]
KLIFLWLLWPVTLACFVLAAV derived from VME1;
iii)[SEQ ID NO: 3]
LPKEITVATSRTLSYYKLGA derived from VME1;
iv)
[SEQ ID NO: 4]
GLEAPFLYLYALVYFLQSINFV derived from AP3A;
v)
[SEQ ID NO: 5]
QMAPISAMVRMYIFFASFYYVWK derived from R1AB;
vi)
[SEQ ID NO: 6]
KVTLVFLFVAAIFYLITPVHVMSK derived from R1AB;
vii)
[SEQ ID NO: 7]
GLVAEWFLAYILFTRFFYVL derived from R1AB;
viii)
[SEQ ID NO: 8]
KRAKVTSAMQTMLFTMLRKL derived from R1A;
ix)
[SEQ ID NO: 9]
EIPVAYRKVLLRKNGNKGAG derived from R1AB;
x)
[SEQ ID NO: 10]
ELSLIDFYLCFLAFLLFLVLIMLII derived from NS7B;
xi)
[SEQ ID NO: 11]
AQFAPSASAFFGMSRIGMEV derived from NCAP;
xii)
[SEQ ID NO: 12]
ALALLLLDRLNQLESKMSGK derived from NCAP;
xiii)
[SEQ ID NO: 13]
VILLNKHIDAYKTFPPTEPK derived from NCAP;
and
xiv)
a polypeptide that is at least 60% identical with
one of the peptides of parts i-xiii.

Yet more preferably still said polypeptide of part xiv) has at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% identity with one of the peptides of parts i)-xiii).

More specifically when treating cancer, particularly melanoma or colorectal cancer, the PeptiBAC ‘platform’ uses tumour associated antigens (TAAs), tumour-specific antigens (TSPs) or neoantigens.

In a preferred embodiment of the invention the cancer antigens are derived from tyrosinase-related protein-2 (Trp2) and/or glycoprotein 100 (gp100) which are endogenously expressed in certain melanomas (e.g. in the model B16.F10 melanoma). Alternatively, the cancer antigens are derived from a modified tumour rejection antigen AH1 that is derived from the gp70 envelope protein of murine leukemia virus (MuLV). Ideally, these forms of PeptiBAC are administered intratumorally.

In a preferred embodiment of the invention said disease is cancer and said peptide antigen comprises at least one of the following peptides, ideally attached covalently or non-covalently onto the bacterial envelope without having been genetically encoded by said BCG bacterial vector:

i)
[SEQ ID NO: 14]
SIINFEKL;
ii)
[SEQ ID NO: 15]
SVYDFFVWL derived from tyrosine related protein 2;
iii)
[SEQ ID NO: 16]
KVPRNQDWL derived from gp100;
iv)
[SEQ ID NO: 56]
SPSYVYHQF a modified sequence derived from tumour
rejection antigen AH1
v)
a polypeptide that is at least 60% identical with
the peptides of parts i, ii, iii or iv.

Yet more preferably still said polypeptide of part v) has at least 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% identity with one of the peptide of parts i), ii) iii) or iv).

BCG bacteria coated with tumour-specific peptides broadens the immune response to include the treatment of a tumour associated with the peptide antigens coated on the modified BCG.

In yet a further preferred embodiment of the invention said peptide is a Pan MHC-II molecule such as PADRE-AKFVAAWTLKAAA [SEQ ID NO:17] whilst this peptide is not infection/tumour related per se, PADRE is a universal T helper epitope that can enhance immune responses elicited by more specific epitopes such as infection or tumour-specific epitopes. Accordingly, its use in working the invention in combination with a further disease specific peptide antigen is favoured.

Advantageously, said peptide antigens can stimulate a peptide-specific immune response in a subject and, more advantageously still, because said peptides have not been genetically encoded by said bacteria, but have been attached to the bacteria covalently or non-covalently using a peptide linker, this attachment can be executed quickly and efficiently. Typically to facilitate attachment of said peptide antigens, said peptide antigen(s) is/are poly-lysine or poly-arginine extended using at least 4, ideally, 5, 6, 7, 8, or 9 lysines or arginines. Most typically 6 lysines are used and attached most preferably at the amino end of the peptide.

Accordingly, the peptides for attachment to the bacteria are selected from the group comprising or consisting of:

[SEQ ID NO: 18 or 48]
KKKKKK(KKK)-IAMACLVGLMWLSYFIASFRLFAR derived from
VME1;
[SEQ ID NO: 19 or 46]
KKKKKK(KKK)-KLIFLWLLWPVTLACFVLAAV derived from
VME1;
[SEQ ID NO: 20 or 47]
KKKKKK(KKK)-LPKEITVATSRTLSYYKLGA derived from
VME1;
[SEQ ID NO: 21 or 60]
KKKKKK(KKK)-GLEAPFLYLYALVYFLQSINFV derived
fromAP3A;
[SEQ ID NO: 22 or 61]
KKKKKK(KKK)-QMAPISAMVRMYIFFASFYYVWK derived from
R1AB;
[SEQ ID NO: 23 or 62]
KKKKKK(KKK)-KVTLVFLFVAAIFYLITPVHVMSK derived from
R1AB;
[SEQ ID NO: 24 or 63]
KKKKKK(KKK)-GLVAEWFLAYILFTRFFYVL derived from
R1AB;
[SEQ ID NO: 25 or 64]
KKKKKK(KKK)-KRAKVTSAMQTMLFTMLRKL derived from
R1A;
[SEQ ID NO: 26 or 65]
KKKKKK(KKK)-EIPVAYRKVLLRKNGNKGAG derived from
R1AB;
[SEQ ID NO: 27 or 66]
KKKKKK(KKK)-ELSLIDFYLCFLAFLLFLVLIMLII derived
from NS7B;
[SEQ ID NO: 28 or 67]
KKKKKK(KKK)-AQFAPSASAFFGMSRIGMEV derived from
NCAP;
[SEQ ID NO: 29 or 59]
KKKKKK(KKK)-ALALLLLDRLNQLESKMSGK derived from
NCAP;
[SEQ ID NO: 30 or 40]
KKKKKK(KKK)-VILLNKHIDAYKTFPPTEPK derived from
NCAP;
[SEQ ID NO: 31 or 42]
KKKKKK(KKK)-SIINFEKL
[SEQ ID NO: 32 or 43]
KKKKKK(KKK)-SVYDFFVWL;
[SEQ ID NO: 33 or 41]
KKKKKK(KKK)-KVPRNQDWL;
[SEQ ID NO: 34 or 39]
KKKKKK(KKK)-AKFVAAWTLKAAA;
[SEQ ID NO: 57 or 58]
KKKKKK(KKK)-SPSYVYHQF;
where KKKKKK(KKK)-is a 6 KKKKKK or 9 KKKKKK(KKK)
amino acid linker linker or where RRRRRR(RRR)-is
a 6 RRRRRR or 9 RRRRRR(RRR) amino acid linker;
and a polypeptide that is at least 60% identical
with one of the afore peptides.

Whilst we had also envisaged working the invention by attaching peptide antigens to said BCG using a Cell-penetrating peptide (CPP) linker, we have surprisingly discovered that CPPs are toxic to BCG, thus reducing its viability and so removing the advantages one seeks when using a live attenuated bacteria such as, generally, the expression of multiple antigens, mass production and the induction of a strong immune response and, specifically (in the context of a cancer therapy), modulation of the tumour microenvironment (TME) by multiple mechanisms including the induction of a massive secretion of chemokines and cytokines that recruit T cells and other immune cells to the TME, polarization of M2 macrophages towards a more M1-like phenotype and enhanced activation and reduced exhaustion of tumour-specific T cells, leading to enhanced effector functions.

In a further preferred embodiment of the invention said modified BCG is coated with a plurality of different peptide antigens, for example, two different peptide antigens, in which case said modified BCG (PeptiBAC) is bivalent. Alternatively still, said modified BCG is coated with three different peptide antigens, in which case said PeptiBAC is trivalent. More alternatively still, said modified BCG is coated with more than three different peptide antigens, in which case said PeptiBAC is polyvalent. Ideally the nature of the different peptide antigens to be used for coating said bacteria are selected having regard to the nature of the result to be achieved and/or the nature of the disease to be treated. Ideally, antigens expressed on the surface of cancer/disease cells are used as peptide antigens; or antigens derived from an infectious agent to be targeted are used as peptide antigens. Additionally, or alternatively, antigens displayed by MHC-I or MHC-II are used. In this way the nature of the immune response to be elicited can be amplified to maximise the effect of the PeptiBAC therapy.

For example, in the context of infection, where only one infection peptide antigen, either a virus associated antigen or even a T helper epitope effective in the treatment of an infection, is used to coat the modified BCG, the PeptiBAC is termed monovalent e.g. PeptiBAC targeting a pan MHC class I or II molecule (herein referred to as PeptiBAC-P).

Where two infection peptide antigens, such as one or two different infection associated antigen(s) and/or one or two different T helper epitope(s) effective in the treatment of infection, to provide a total of two different antigens/epitopes, are used to coat the modified BCG, the PeptiBAC is termed bivalent.

Where three infection peptide antigens, such as one, two or three different infection associated antigens and/or one, two or three different T helper epitopes, to provide a total of three different antigens/epitopes, are used to coat the modified BCG, the PeptiBAC is termed trivalent.

Similarly, for example, in the context of cancer, where only one cancer peptide antigen, either a tumour associated antigen or even a T helper epitope effective in the treatment of cancer, is used to coat the modified BCG, the PeptiBAC is termed monovalent e.g. PeptiBAC targeting a pan MHC class I or II molecule (herein referred to as PeptiBAC-P).

Where two cancer peptide antigens, such as one or two different tumour associated/tumour rejection antigens and/or one or two different T helper epitope(s) effective in the treatment of cancer, to provide a total of two different antigens/epitopes, are used to coat the modified BCG, the PeptiBAC is termed bivalent e.g. PeptiBAC targeting Trp2 and gp100 (herein referred to as PeptiBAC-TG).

Where three cancer peptide antigens, such as one, two or three different tumour associated/tumour rejection antigens and/or one, two or three different T helper epitopes, to provide a total of three different antigens/epitopes, are used to coat the modified BCG, the PeptiBAC is termed trivalent PeptiBAC e.g. targeting Trp2, gp100 and pan MHC class I or II molecules (herein referred to as PeptiBAC-TGP).

Whilst the invention can be practised using any strain of BCG, in a preferred embodiment of the invention BCG strain BCG-Russia, and/or BCG-Danish strain 1331 and/or BCG-Bulgaria are used. However, those skilled in the art will appreciate the modifications between strains are small and extremely unlikely to effect the working of the invention.

The distinguishing characteristic of BCG, a bacterium belonging to the Mycobacterium genus, is a complex cell envelope containing the inner plasma membrane (IM), the peptidoglycan—arabinogalactan complex, and the outer membrane (OM) that is covalently linked to the arabinogalactan. It is to this cell envelope that the peptides are attached.

In yet a further preferred embodiment of the invention said disease is selected from the list comprising: an infectious disease, a respiratory disease, influenza, tuberculosis TB, common cold, a coronavirus infection comprising SARS and MERS, an autoimmune disease and cancer.

In yet a still further preferred embodiment of the invention said bacteria is further modified to include any one or more of the following features, including any and all combinations thereof.

In a preferred embodiment said modified bacteria comprises the insertion of at least one transgene that encodes a co-stimulatory molecule and, ideally, two transgenes wherein one of said genes leads to activation of the innate immune system and the other leads to activation of the adaptive immune system. Preferred transgenes include CD40L for activating the innate immune system by the use of antigen presenting cells (APCs) to drive CD8+ T-cell responses and OX40L for activating the adaptive immune system by increasing clonal expansion, survival of CD8+ T-cells and the formation of a large pool of memory T-cells.

In the alternative, DNA encoding OX40L and CD40L may be joined and inserted as a fusion molecule using known genetic engineering techniques. Typically, CD40L is inserted immediately downstream from OX40L but it is possible to work the invention with the reverse configuration.

The BCG utilized in the present invention may also comprise other modifications than those described above. Any additional components or modifications may optionally be used but are not obligatory for the present invention.

It follows that the bacteria of the invention has been engineered to stimulate an immune response against a disease such as an infection thus acting as a vaccine or a treatment. It also follows from the above that the bacteria of the invention has been engineered to stimulate an immune response against a disease such as cancer and specifically in a tumour environment where, typically, the immune system is compromised by the evasive mechanisms employed by the cancer cells.

The elegance of this modified BCG platform technology is the introduction of disease and immunity-inducing peptides non-genetically onto the BCG vaccine, which makes this approach highly adaptable and thus suitable for personalized immunotherapeutic approaches that rely on the identification of patient-specific neo-antigens.

Accordingly, the invention extends to a pharmaceutical composition comprising at least one modified BCG of the invention and a suitable carrier. In a preferred embodiment of the invention said pharmaceutical composition is formulated for intradermal, intranasal, subcutaneous, percutaneous, intratumoral, intramuscular, intra-arterial, intravenous, intrapleural, intravesicular, intracavitary, peritoneal injection, or oral administration.

Accordingly, in yet a further aspect the invention concerns a method of treating a disease in an individual comprising administering to the individual an effective amount of the modified BCG according to the invention or a pharmaceutical composition comprising at least one modified BCG according to the invention.

Given tumors have evolved several immunosuppressive mechanisms to counteract the immune cells of the body, the therapy of the invention is also ideally practised in combination with the use of a checkpoint molecule. The best characterized checkpoint pathways are cytotoxic T-lymphocyte protein 4 (CTLA-4) and programmed cell death protein 1 pathway (PD-1/PD-L1). Thus, the modified BCG of the invention can be utilized in combination with a checkpoint modulator or immune checkpoint inhibitor such as anti-PD1, anti-PD-L1 or anti-CTLA-4 molecules to counteract the immunosuppressive tumor environment and to cause a strong anti-immune response.

The modified BCG acts as an active adjuvant because it provides the danger signal required for an optimal immune response against a target peptide. The oncolytic cell killing is immunogenic by nature, which causes changes in the tumour micro-environment that are likely to strengthen the immune response to the peptides/tumour. Therefore, using our modified BCG: co-administered with or physically complexed with immunomodulatory peptides results in a superior anti-tumor immune response when compared to either peptide vaccines or BCG vaccines alone.

In a preferred method of the invention the administration of said modified BCG is preceded by and/or followed by the administration of a checkpoint modulator molecule or an immune checkpoint inhibitor molecule. Alternatively still, said modified BCG is co-administered with a checkpoint modulator molecule or an immune checkpoint inhibitor molecule.

Accordingly, in a further aspect of the invention there is provided a combination therapeutic comprising the modified BCG according to the invention and at least one checkpoint molecule, such as: cytotoxic T-lymphocyte protein 4 (CTLA-4) or programmed cell death protein 1 pathway (PD-1/PD-L1).

Additionally, or alternatively still, the invention concerns at least one modified BCG or pharmaceutical composition according to the invention for use in treating a disease as herein described.

Additionally, or alternatively, the invention concerns the use of at least one modified BCG according to the invention in the manufacture of a medicament to treat a disease as herein described.

Most preferably the cancer referred to herein includes any one or more of the following cancers: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, oligodendroglioma, neuroblastoma, meningioma, spinal cord tumor, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, esophagus cancer, gall bladder cancer, head cancer, eye cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, multiple myeloma, ovarian cancer, endocrine pancreatic cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

Most preferably the infection referred to herein includes any one or more of the following infections: a respiratory disease, influenza, tuberculosis TB, common cold or a coronavirus infection comprising SARS and MERS.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Throughout the description and claims of this specification, the singular encompasses the plural unless the context otherwise requires. In particular, where the indefinite article is used, the specification is to be understood as contemplating plurality as well as singularity, unless the context requires otherwise.

An embodiment of the present invention will now be described by way of example only with reference to the following wherein:

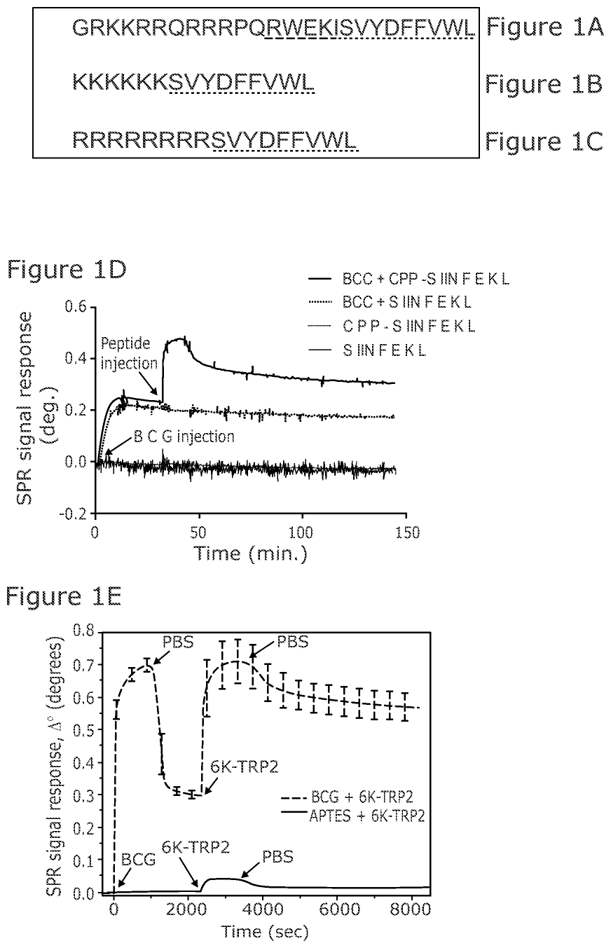

FIGS. 1A-1C show a schematic presentation of A) an N-terminal cell penetrating peptide-containing immunomodulatory peptide, B) an N-terminal polylysine-containing immunomodulatory peptide and C) a polyarginine-containing immunomodulatory peptide. Color code for different functional sequences of the entities: In blue: cell penetrating peptide sequence (A) or polylysine sequence (B). In green: immunoproteasome processing site. In orange: the immunomodulatory peptide or MHC-I restricted epitope.

FIGS. 1D-1E show surface plasmon resonance (SPR) measurements to confirm high affinity of cell penetrating peptide (CPP)-containing immunomodulatory peptides [A] and polylysine (6K) linker-containing immunomodulatory peptides [B] to the BCG bacterial surface. Immunomodulatory peptides containing neither CPP nor 6K do not interact with the bacterial surface.

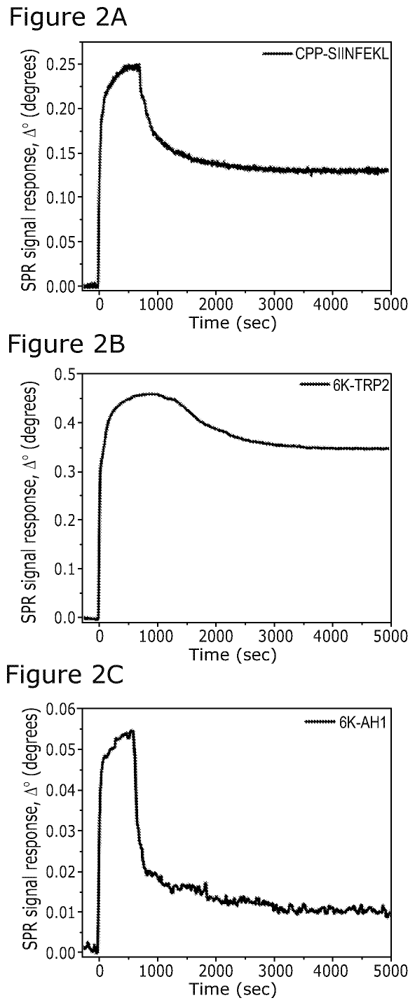

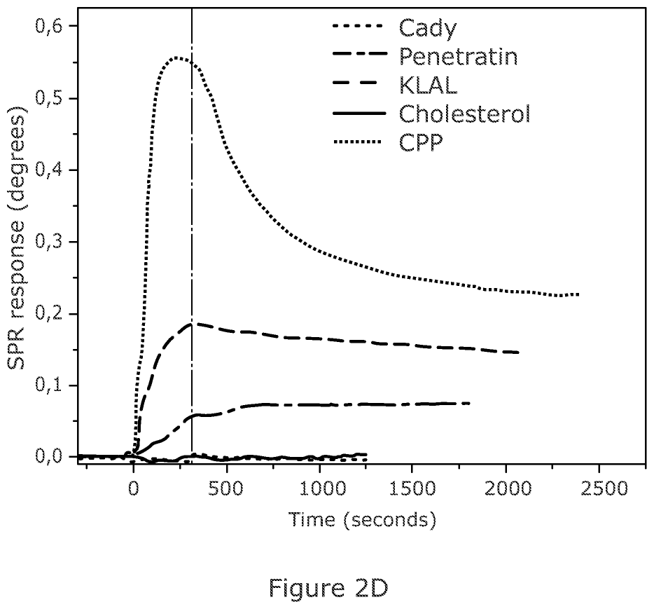

FIGS. 2A-2C show surface plasmon resonance (SPR) analysis of the peptide/BCG interaction.
A) Surface plasmon resonance analysis of the interaction between the CPP-OVA and BCG.
B) Surface plasmon resonance analysis of the interaction between the polyK-Trp2 and BCG.
C) Surface plasmon resonance analysis of the interaction between the polyK-AH1 and BCG.

FIG. 2D shows surface plasmon resonance (SPR) analysis of various attachment moieties used in coating of BCG. Various CPP sequences as well as cholesterol moiety were tested by surface plasmon resonance (SPR) for their efficacy at anchoring therapeutic peptides into the mycobacterial cell wall. Cady sequence GLWRALWRLLRSLWRLLWRA (SEQ ID NO 35), Penetratin sequence RQIKIWFQNRRMKWKK (SEQ ID NO 36), KLAL sequence KLALKLALKALKAALKLA (SEQ ID NO 37), N-terminal cholesterol moiety and CPP Tat sequence GRKKRRQRRRPQ (SEQ ID NO 38) were compared for binding efficacy.

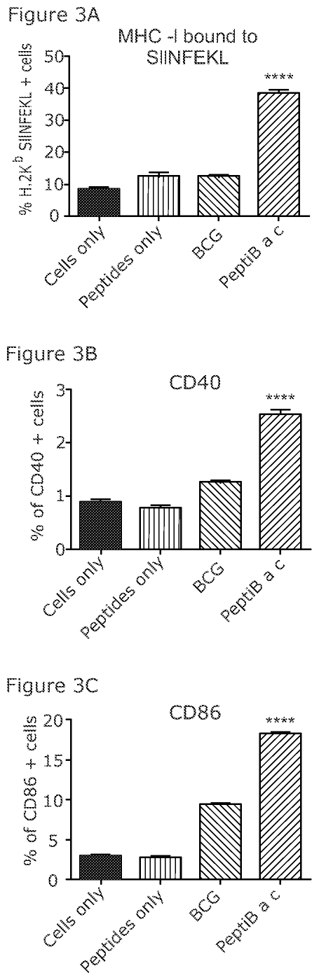

FIGS. 3A-3C show the modified BCG of the invention, hereinafter referred to as PeptiBAC, can efficiently deliver immunomodulatory CPP-containing peptides into dendritic cells (DCs) allowing DCs to present these peptides in major histocompatibility complexes [A]. PeptiBAC can induce DC maturation and activation as measured by the expression of CD40 [B] and CD86 [C] surface markers.

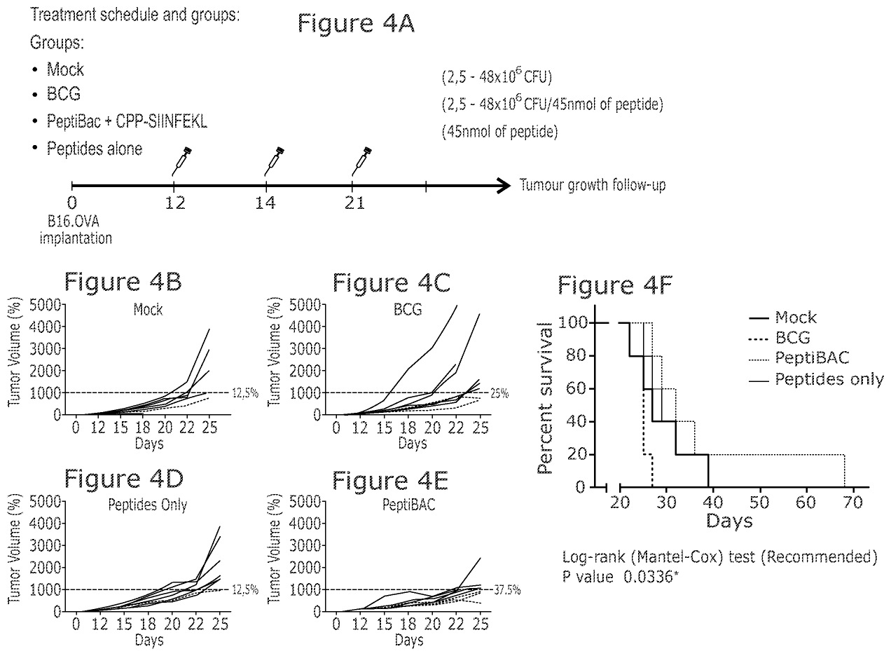

FIGS. 4A-4F show in (A) the upper part the treatment schedule and groups and the lower panels show PeptiBAC therapy decrease tumour growth control in murine model of melanoma. (B-E) show individual growth curves of each treated mice. Responders in each group are shown in green/light grey. The percentage of responders is shown on the right of the dotted line in each panel. (F) shows Kaplan-Meier survival curve for each group. Use of peptiBAC almost doubles survival.

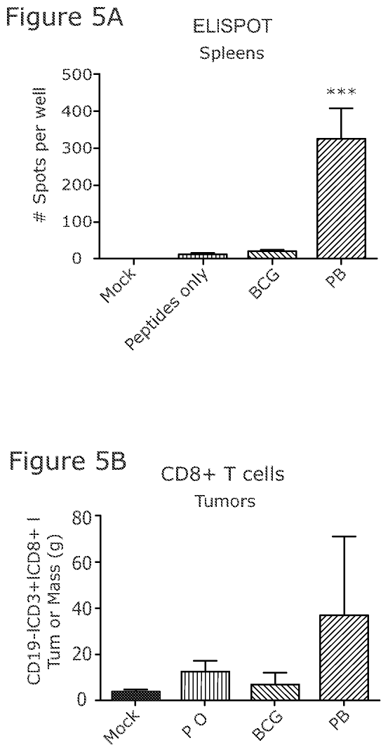

FIGS. 5A-5B show systemic peptide-specific T cell response elicited by the PeptiBAC (PB, BCG complexed with CPP-containing SIINFEKL peptide) platform as measured by the ELISpot assay from [A] the spleens of treated animals and [B] increased CD8+ T cell influx into the tumour microenvironment in PB group as compared to peptides only (PO) or BCG groups.

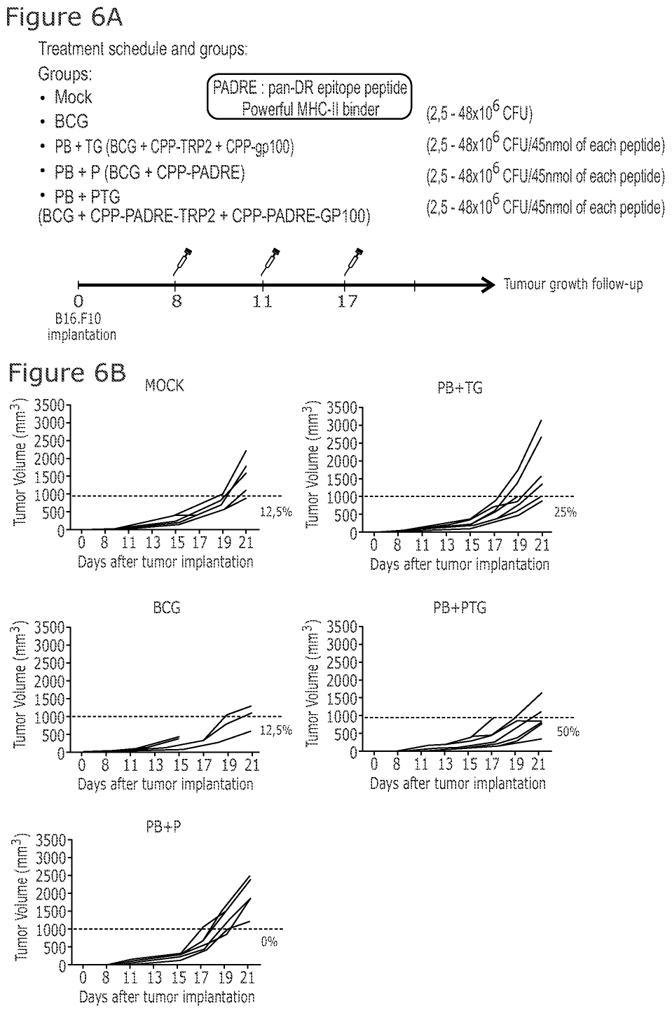

FIGS. 6A-6B show schedule and treatment groups [A] in animal model of highly immunosuppressive mouse melanoma B16.F10 and [B] individual tumour growth curves of the treated animals with the response rates shown under the blue line in each group.

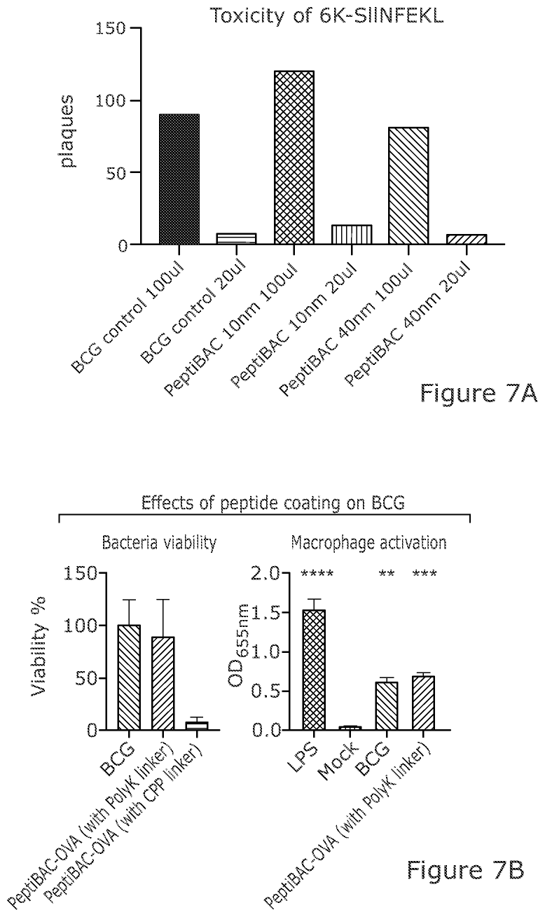

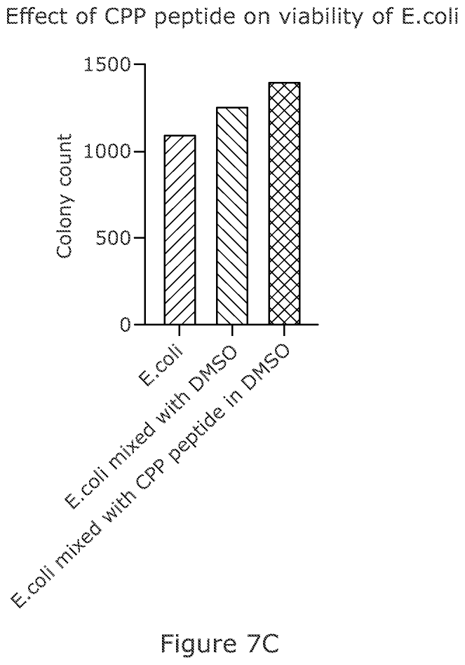

FIG. 7A show bacterial plaque formation after BCG formulation with different amounts of polylysine linker containing immunomodulatory peptide. BCG was complexed with 10 nm and nm of the immunomodulatory peptide containing polylysine linker as the bacterial membrane attachment moiety. Polylysine linker attached peptides do not affect the viability of the BCG bacteria.

FIG. 7B shows that coating BCG with CPP-containing peptide antigen but not with poly-lysine-containing peptide antigen decrease BCG viability. BCG was coated with either CPP-containing peptide antigen (CPP-OVA) or poly-lysine-containing antigen (polyK-OVA) and complexes were directly plated for colony formation. RAW-Blue cells (100.000 cells/well) were stimulated with BCG or PeptiBAC-OVA (using PolyK-OVA peptide) and the NF-kB/AP-1 activation was measured 24 hours post-infection.

FIG. 7C shows coating E. coli (a Gram negative bacteria) with CPP-containing peptide does not affect bacterial viability. E. coli was coated with 14 nmol of CPP-containing peptide antigen (CPP-Trp2) and complexes were directly plated for colony formation.

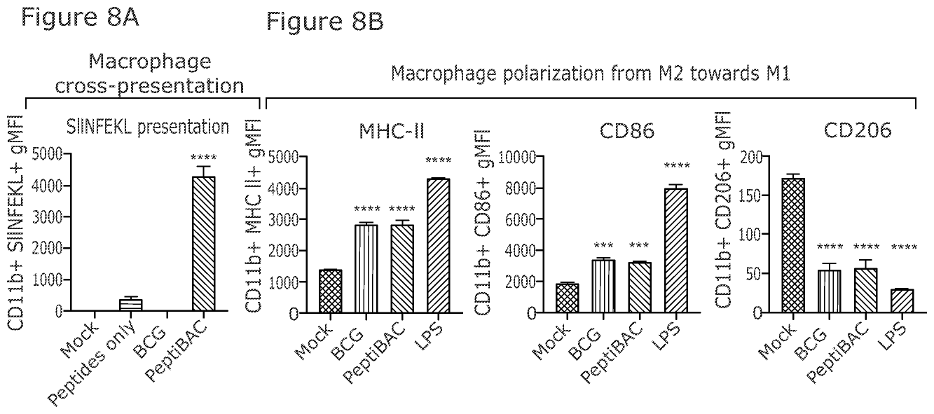

FIGS. 8A-8B show macrophages can cross-present antigens delivered by the PeptiBAC platform and can be polarized towards M1-like phenotype. A) Mouse bone-marrow derived macrophages were pulsed with PeptiBAC-OVA, BCG, poly-lysine-containing SIINFEKL peptide alone or left un-pulsed (Mock). Cross-presentation was determined by flow cytometry using APC-conjugated anti-H-2Kb bound to SIINFEKL. B) M2 macrophages were treated 24 h with BCG, PeptiBAC-OVA, LPS (10 ug/ml) or left untreated (Mock). MHC-II, CD86 and CD206 expression was determined by flow cytometry. Each bar is the mean±SEM of technical triplicates. Statistical analysis was performed with one-way ANOVA. **** p<0.0001 *** p<0.001.

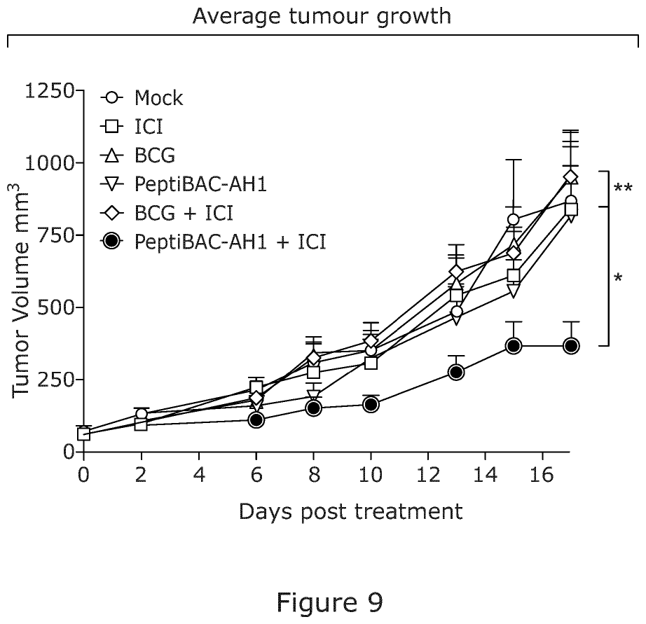

FIG. 9 shows average tumour growth curves for each treatment group in CT26 colon carcinoma experiment. PeptiBAC-AH1 (BCG coated with polyK-containing AH1 epitope peptide), ICI (anti-PD-1 immune checkpoint inhibitor). Statistical analysis was performed with two-way ANOVA. * p<0.05, ** p<0.01

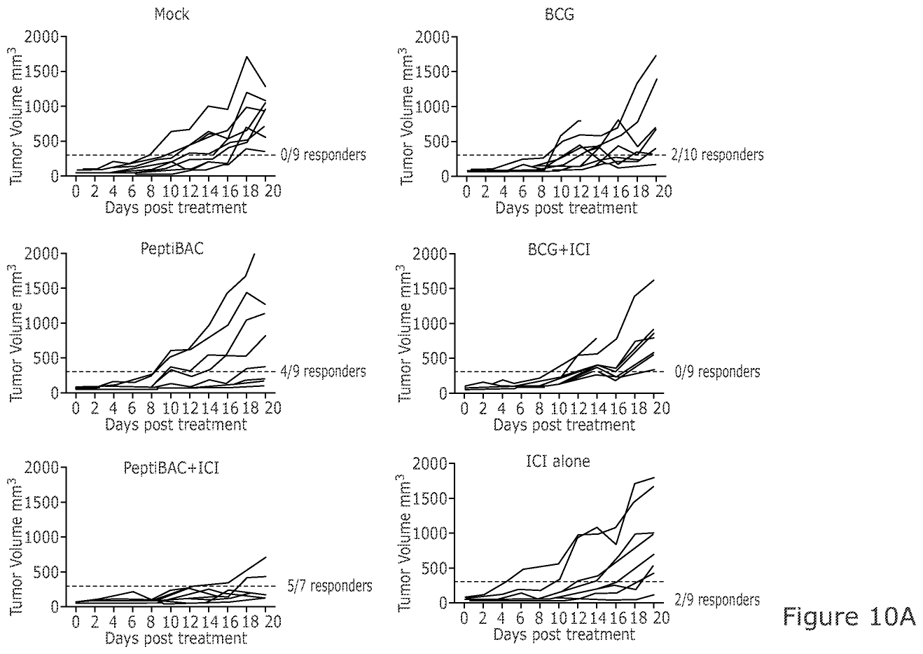

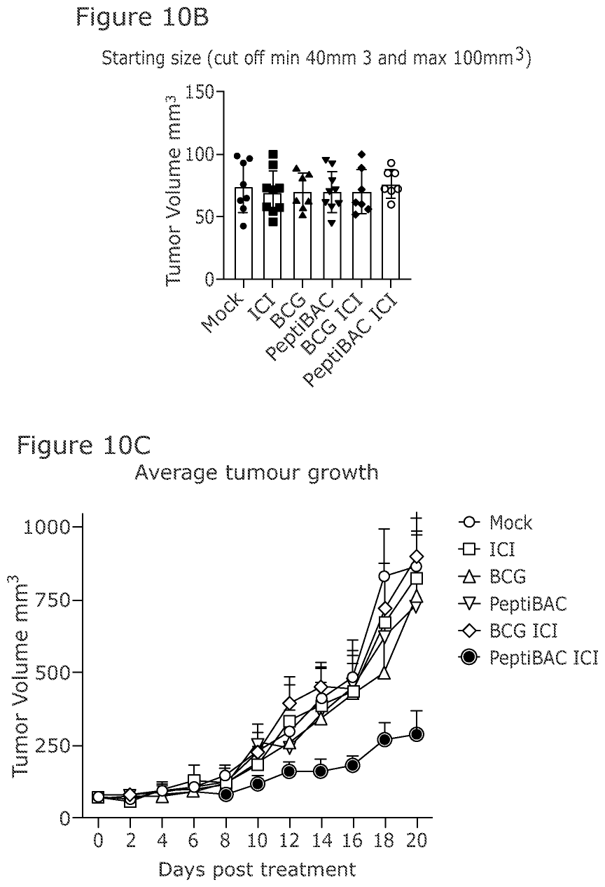

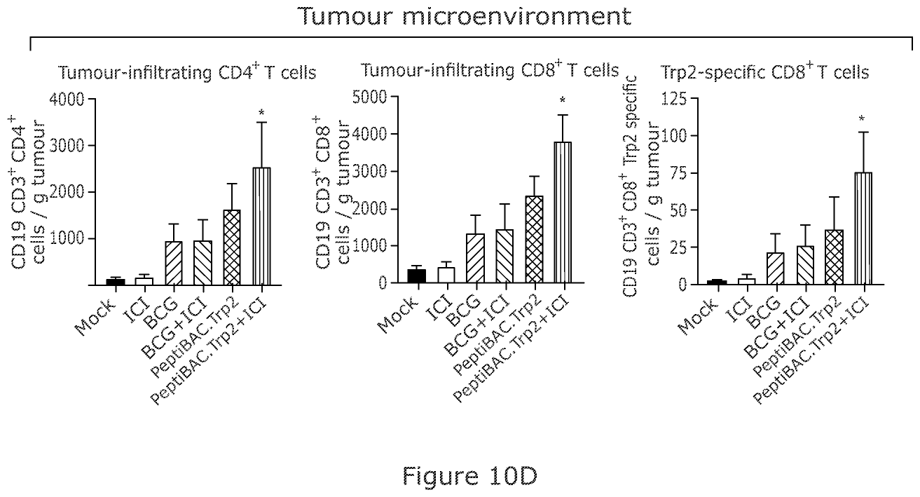

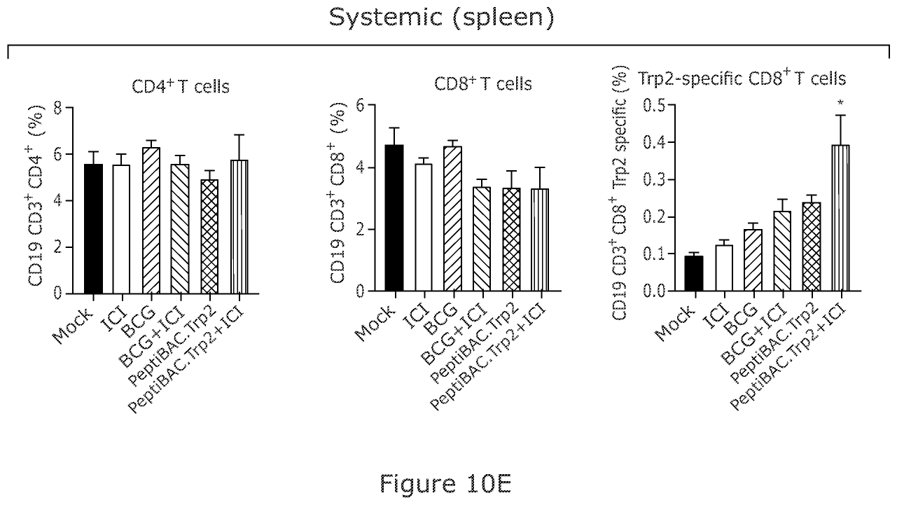

FIGS. 10A-10C show PeptiBAC-Trp2 (BCG coated with polyK-containing Trp2 epitope peptide) treatment enhances the response rate to checkpoint inhibitor therapy. Panel [A] shows individual growth curves of each treated mice. The percentage of responders is shown on the right of the dotted line in each panel. Panel [B] shows the starting size of the treated tumours in each group. Panel [C] shows average tumour growth of each group

FIGS. 10D-10E shows PeptiBAC-Trp2 (BCG coated with polyK-containing Trp2 epitope peptide) in combination with anti-PD1 induces robust infiltration of tumour-specific CD8+ T cells into the tumour in a syngeneic mouse model of B16.F10.9/K1 melanoma. A) Immunological analysis of tumours of treated mice. B) Immunological analysis of spleens of treated mice. The number of mice in each group was 9-11. Statistical analysis was performed with one-way ANOVA. * p<0.05.

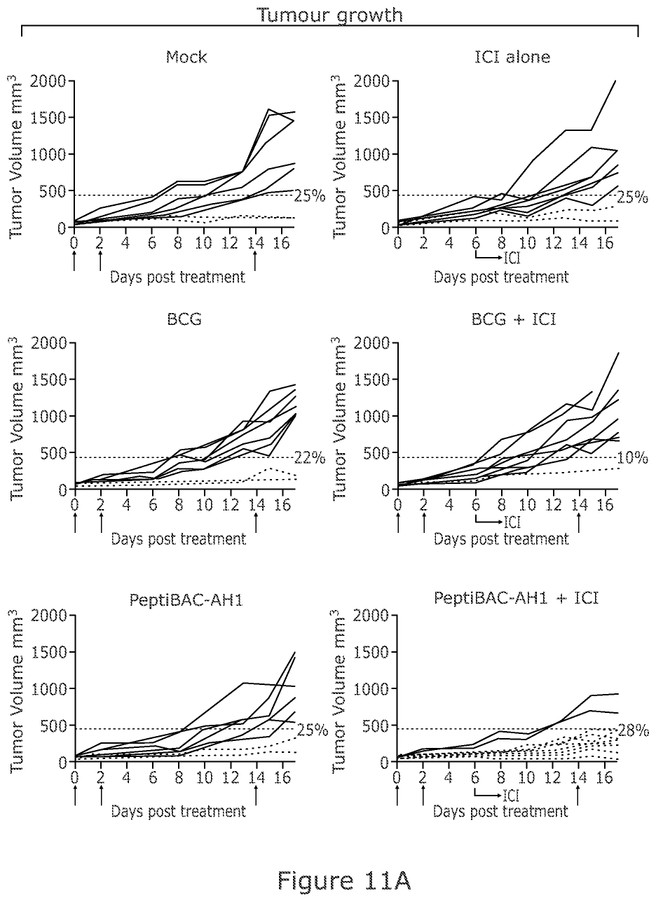

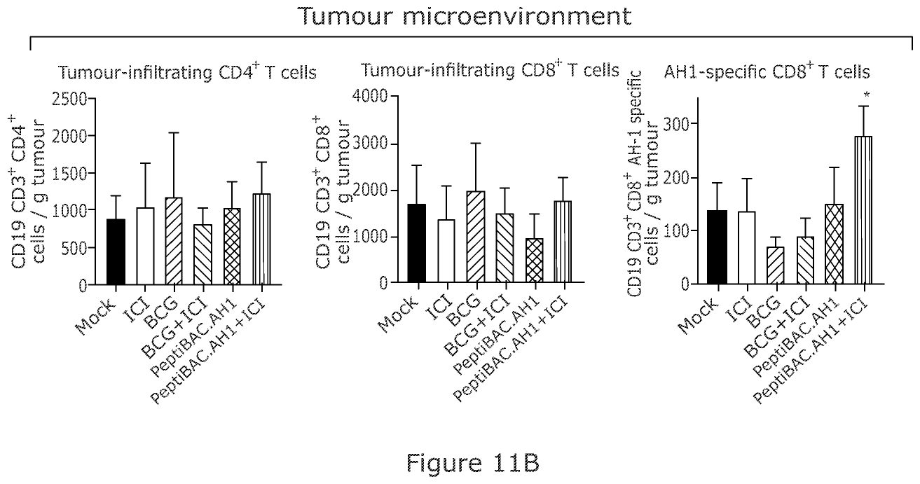

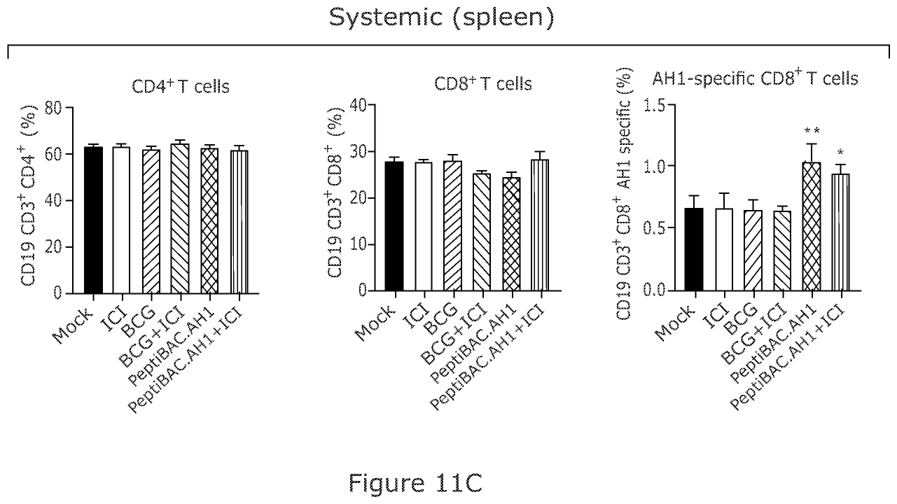

FIGS. 11A-11C show PeptiBAC-AH1 (BCG coated with polyK-containing AH1 epitope peptide), in combination with anti-PD1 improves tumour growth control compared to either monotherapies and induces systemic tumour-specific CD8+ T cell response and robust infiltration of tumour-specific CD8+ T cells into the tumour in a syngeneic mouse model of CT26 colorectal cancer. A) Anti-PD-1 immune checkpoint inhibitor alone (100 μg/dose given intraperitoneally three times a week, starting at day 6), BCG alone or in combination with anti-PD-1 immune checkpoint inhibitor and PeptiBAC-AH1 alone or in combination with anti-PD-1 immune checkpoint inhibitor was given intratumorally 11-, 13-, and 25-days post tumour implantation. Individual tumour growth curves for all treatment groups are shown. A threshold of 450 mm³ was set to define the percentage of mice responding to the different therapies (dotted line). The percentage of responders in each treatment group is shown on the right side of the dotted line. B and C) Immunological analysis of tumours and spleens of treated mice. The number of mice in each group was 8-10. Statistical analysis was performed with one-way ANOVA. * p<0.05, ** p<0.01.

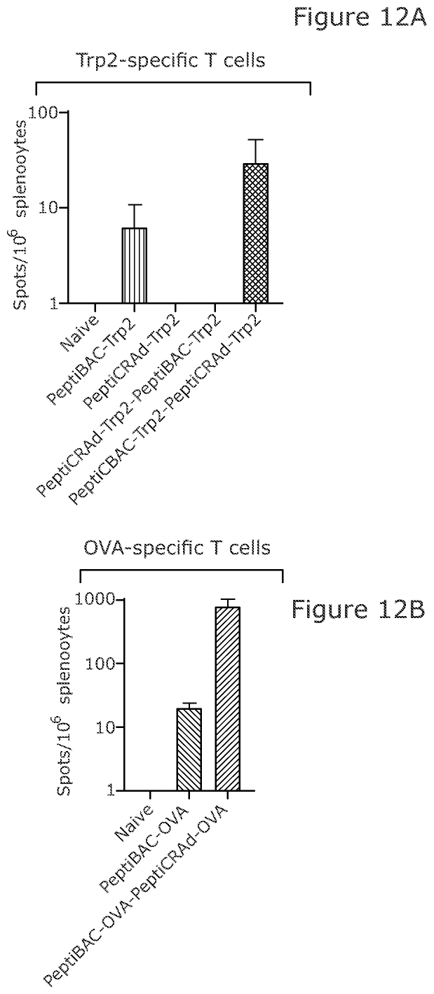

FIGS. 12A-12B show heterologous prime-boost vaccination with PeptiCRAd platform improves peptide-specific T cell responses elicited by the PeptiBAC platform. A) Naïve C57BL/6JOlaHsd immunocompetent mice were vaccinated subcutaneously with 1×10⁹ VP/dose of PeptiCRAd-Trp2 or 2-8×10⁶ C.F.U/dose of PeptiBAC-Trp2 (BCG coated with polyK-containing Trp2 epitope peptide) or saline as a mock-treated group. Prime and boost vaccinations were performed 14 days apart and 4 days after the boost, mice were sacrificed, and spleens were collected for enzyme-linked immunospot (ELISPOT) assay. The number of mice in each vaccination group was 4, and in control group not receiving vaccinations the number of mice was 2. B). Similarly to A, mice were vaccinated with PeptiBAC-OVA (BCG coated with polyK-containing SIINFEKL epitope peptide) or PeptiBAC-OVA followed by PeptiCRAd-OVA booster. The number of mice in each vaccination group was 5.

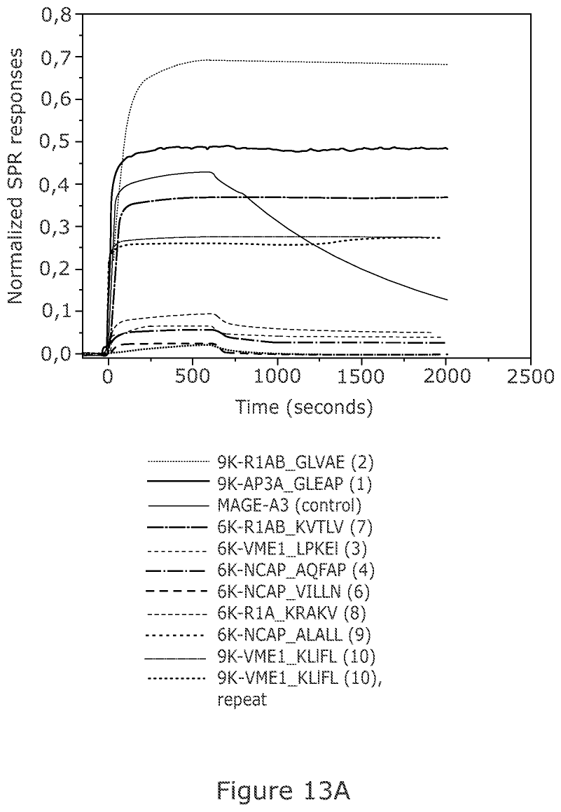

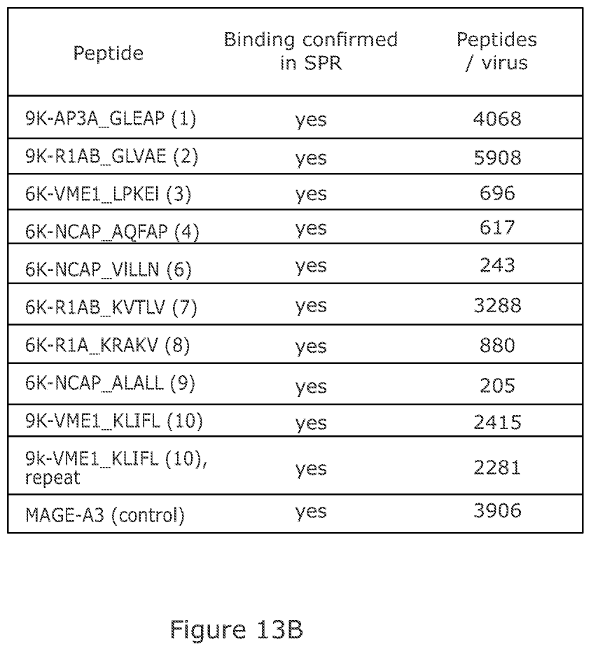

FIGS. 13A-13B show Surface Plasmon Resonance (SPR) analysis was done to evaluate peptide binding properties to viral capsid. Electrostatic interaction between SARS-CoV2-derived peptides (20 to 24 amino acids in length) and human Adenovirus capsid is presented. These data demonstrate that all nine selected peptides for investigation can be electrostatically attached to the capsid of Adenovirus. MAGE-A3 peptide (20 aa) was used as a positive control.

FIG. 14 shows a Interferon-gamma ELISPOT assay to demonstrate a strong T-cell response against SARS-CoV2-derived peptides in Peripheral blood mononuclear cells (PBMCs) isolated from COVID convalescents. PBMCs from 9 patients treated in intensive care (ICU) due to severe COVID were isolated. PBMCs were collected at 6 months from the internalization (long term hospitalized). These data demonstrate that all nine selected peptides are clinically relevant since they can each trigger a cytotoxic T-cell responses in SARS-CoV2 infected patients. As a control, PBMCs from healthy donors were used and T-cell responses against the same SARS-CoV2-derived peptides were assessed.

Rationale: The subject that ended up in the hospital might be the more susceptible. We could justify the selection of this cohort and this time point by underlining that the responses of these people are the most important.

Specific Description

Materials and Methods:

Proof of Concept

The infectious disease peptide antigens described herein could not be tested in an animal model because they are biologically adapted for infection in humans. Accordingly, the use of BCG to deliver immunologically effective, surface linked peptide antigens was shown to work using BCG coated with cancer peptide antigens. This modified BCG was investigated for immune activity using both existing murine cell lines and murine strains.

Peptides:

Peptides used in this study are listed below and were purchased from PepScan and Ontores:

CPP peptides:
[SEQ ID NO: 49]
GRKKRRQRRRPQRWEKISIINFEKL
[SEQ ID NO: 50]
GRKKRRQRRRPQRWEKISVYDFFVWL
[SEQ ID NO: 51]
GRKKRRQRRRPQRWEKIKVPRNQDWL
[SEQ ID NO: 52]
GRKKRRQRRRPQRRAKFVAAWTLKAAA
[SEQ ID NO: 53]
GRKKRRQRRRPQRRAKFVAAWTLKAAAKVPRNQD
[SEQ ID NO: 54]
GRKKRRQRRRPQRRAKFVAAWTLKAAASVYDFFVWL
(SEQ ID NO 35)
GLWRALWRLLRSLWRLLWRA, Penetratin sequence
(SEQ ID NO 36)
RQIKIWFQNRRMKWKK, KLAL sequence
(SEQ ID NO 37)
KLALKLALKALKAALKLA, N-terminal cholesterol moiety
and
(SEQ ID NO 38)
GRKKRRQRRRPQ CPP, Tat sequence
Polylysine peptides:
(SEQ ID NO: 42)
KKKKKKSIINFEKL
(SEQ ID NO: 43)
KKKKKKSVYDFFVWL
[SEQ ID NO: 58]
KKKKKK SPSYVYHQF
Other peptides:
[SEQ ID NO: 55]
RWEKISIINFEKL
[SEQ ID NO: 14]
SIINFEKL
[SEQ ID NO: 56]
SPSYVYHQF
[SEQ ID NO: 15]
SVYDFFVWL

Cell Lines

Murine melanoma cell lines B16.OVA, B16.F10 and B16.F10.K1 were cultured in DMEM with 10% foetal calf serum (FBS) (Life Technologies), 1% L-glutamine and 1% penicillin/streptomycin at 37° C./5% CO2. Human triple negative breast cancer cell line MDMBA436 was cultured in RPMI with 10% foetal calf serum (FBS) (Life Technologies) 1% L-glutamine and 1% penicillin/streptomycin at 37° C./5% CO2. Murine DC line Jaws II was cultured in alpha minimum essential medium with 20%

FBS (Life Technologies), ribonucleosides, deoxyribonucleosides, 4 mM L-glutamine (Life Technologies), 1 mM sodium pyruvate (Life Technologies), and 5 ng/mL murine GM-CSF (PeproTech, USA) at 37° C./5% CO₂.

Murine colon carcinoma CT26.wt cell line was purchased from ATCC and was cultured in high glucose RPMI with 10% foetal calf serum (FBS) (Life Technologies), 1% L-glutamine and 1% penicillin/streptomycin. B16F10.9/K1 cell line was kindly provided by Ludovic Martinet (Inserm, France) and was cultured in high glucose DMEM supplemented with 10% FBS, 1% L-glutamine and 1% penicillin/streptomycin. The cell line B16.OVA, a mouse melanoma cell line expressing chicken ovalbumin (OVA), was kindly provided by Prof. Richard Vile (Mayo Clinic, Rochester, MN, USA). B16.OVA cells were cultured in DMEM with 10% FBS (Life Technologies), 1% L-glutamine, 1% penicillin/streptomycin and 5 mg/mL of geneticin. Murine dendritic cell line JAWSII was purchased from ATCC and was cultured in alpha minimum essential medium with 20% FBS (Life Technologies), ribonucleosides, deoxyribonucleosides, 4 mM L-glutamine (Life Technologies), 1 mM sodium pyruvate (Life Technologies), and 5 ng/ml murine GM-CSF (PeproTech, USA). Murine macrophage reporter cell line RAW-Blue (InvivoGen) was cultured in DMEM supplemented with 10% FBS, 1% L-glutamine, 1% penicillin/streptomycin, 100 μg/ml Normocin (InvivoGen) and 100 μg/ml Zeocin (InvivoGen) as a selective antibiotic. Human lung carcinoma A549 cell line was purchased from NIH and was cultured in OptiPRO™ SFM supplemented with 10% FBS (Life Technologies), 1% L-glutamine and 1% penicillin/streptomycin. All cells were cultured at 37° C./5% CO2 and were routinely tested for mycoplasma contamination using a commercial detection kit (Lonza).

BCG Vaccine Preparations:

BCG vaccine preparations were either purchased from InterVax Ltd (Canada) (BCG vaccine for tuberculosis, the BCG-Bulgaria strain), or purchased from AJVaccines (Denmark) (BCG vaccine for tuberculosis, the Danish strain 1331) or were given by Serum Institute of India (India) (BCG vaccine, the BCG-Russia strain, for tuberculosis and ONCO-BCG preparation used in the treatment of bladder cancer).

SII BCG (2-8×106 colony forming units [C.F.U]/vial) and SII-ONCO-BCG vaccine (1-19.2×10⁸ C.F.U/vial), were kindly provided by the Serum Institute of India (Pune, India). BCG vaccine (1.5-6.0×10⁶ C.F.U/vial) was purchased from InterVax (Toronto, Canada), while BCG vaccine AJV (2-8×10⁶ C.F.U/vial) from AJ Vaccines (Copenhagen, Denmark) was a kind gift from Professor Helen McShane (University of Oxford).

Viruses:

An adenovirus expressing murine OX40L and CD40L (VALO-mD901) was used in heterologous prime-boost experiments. The development of VALO-mD901 has previously been described (Ylōsmäki&Ylōsmaki et al. 2021). Briefly, a part of the E3B-region of pAd5/3-D24 backbone plasmid was replaced with human cytomegalovirus (CMV) promoter region, murine OX40L, 2A self-cleaving peptide sequence, murine CD40L gene and rabbit beta-globin polyadenylation signal. The virus was amplified in A549 cells and purified on double caesium chloride gradients and stored below −60° C. in A195 adenoviral storage buffer 16. The viral particle (VP) concentration was measured at 260/280 nm and infectious units (IU) were determined by immunocytochemistry (ICC) by staining the hexon protein on A549-infected cells.

Peptides

GRKKRRQRRRPQRWEKISIINFEKL (SEQ ID NO 49), RWEKISIINFEKL (SEQ ID NO 55), KKKKKK-SIINFEKL (SEQ ID NO 42) and SIINFEKL (SEQ ID NO 14) (containing an MHC class I-restricted epitope from chicken ovalbumin, OVA257-264), KKKKKK-SVYDFFVWL (SEQ ID NO 43) and SVYDFFVWL (SEQ ID NO 15) (containing an MHC class I-restricted epitope from tyrosinase-related protein 2, Trp2180-188), KKKKKK-SPSYAYHQF (SEQ ID NO 44) and SPSYAYHQF (SEQ ID NO 45) (containing a modified MHC class I-restricted epitope from murine leukaemia virus envelope glycoprotein 70 [gp70423-431] where V5A change was made to the original AH1 epitope for enhanced immunogenicity). All peptides were purchased from Zhejiang Ontores Biotechnologies (Zhejiang, China).

PeptiBAC Complex Formation

0.75×10⁵-12×10⁷ C.F.U of BCG resuspended in PBS were complexed with 40-90 nmol of CPP or poly-K (e.g., 6K) peptides resuspended in DMSO and incubated for 15 minutes at RT. After complexation, PeptiBAC complexes were pelleted by centrifugation at 1000 g for 10 min at RT and the buffer was changed to remove unbound peptides.

PeptiCRAd Complex Formation

PeptiCRAd complexes were prepared by mixing VALO-mD901 adenovirus (in A195 storage buffer) and polyK-extended Trp2 epitope (in 0.9% saline) at a ratio of 1.8×10⁵ peptides per one virus particle. The mixture was then incubated at room temperature for 15 min. For animal injections, the complexes were diluted further with 0.9% saline to administration volume.

Surface Plasmon Resonance

Measurements were performed using a multi-parametric SPR Navi 220A instrument (Bionavis, Tampere, Finland). PBS (pH 7.4) was used as a running buffer. A constant flow rate of 20 mL/min was used throughout the experiments, and temperature was set to +20° C. Laser light with a wavelength of 670 nm was used for surface plasmon excitation. A sensor slide with a silicon dioxide surface was activated by 5 min of plasma treatment followed by coating with APTES ((3-aminopropyl)triethoxysilane) by incubating the sensor in 50 mM APTES in isopropanol for 4 hr. The sensor was then washed and placed into the SPR device, and bacteria were immobilized in situ on the sensor surface of the test channel by injecting BCG preparation in PBS (pH 7.4) for 12 min, followed by a wash with PBS. CPP-containing immunomodulatory peptide, polylysine-containing immunomodulatory peptide or peptide without CPP or polylysine sequence (a non-interacting control) were then injected separately into the flow channels of the flow cell.

For testing the interaction between various peptides and the mycobacterial outer membrane, 100 μM of the tested peptides extended with CPP or poly-lysine sequences, or without the attachment moieties (as non-interacting controls) were injected into a BCG coated channel and into an uncoated channel of the flow cell.

The number of peptides per BCG particle were estimated according to the following procedure:
1) First, it was assumed that a fully covered sensor surface forms a monolayer of hexagonally packed layer of BCG particles. This means that only 74% of the sensor surface can be covered by the bacteria (based on geometrical calculations). For this, the average length (2.36 μm) and width (0.47 μm) of a BCG bacterium was converted to a spherical particle with a volume of 0.3887 μm³ and a diameter of 905.5 nm.
2) In order to estimate the thickness of a hexagonally packed layer of BCG particles, we performed optical modelling of the SPR sensor properties for a plain sensor without BCG and a sensor fully covered with a layer of BCG particles. However, in optical modelling of the SPR sensor properties, we needed to consider that the models assume even homogeneous layers without spaces and thus we converted the volume of a sphere to the corresponding value of a cube by using a conversion factor of 0.524 (based on geometrical calculations).
3) In order to estimate the theoretical even homogeneous thickness of a fully covered hexagonally packed BCG layer, we first multiplied the average diameter of BCG with 0.74 (contribution from hexagonal packing) and then with 0.524 (contribution of filling the gaps between spheres into a homogeneous even layer).
4) In this way we obtained a theoretical even homogeneous thickness of a fully covered hexagonally packed layer for BCG of 351.1 nm (assuming an average diameter of 905.5 nm).
5) Hereafter, we calculated through optical modelling the maximum SPR angular response induced by this BCG layer by assuming a refractive index of 1.35 for BCG and obtained 2.28° (see supplementary FIG. 7.).
6) The actual measured SPR response during immobilization of the BCG on the SPR sensor surface was then divided with the corresponding maximum SPR angular response modelled for a monolayer of hexagonally packed layer of BCG (i.e. 2.28°). This ratio was then assumed to reflect the percentage of the detection area covered with BCG. For the measurements for the different peptides used in this study the corresponding percentages were: 22.6% (6K-AH1 peptide), 13.6% (6K-TRP2) and 11.0% (CPP-SIINFEKL).
7) As the detection area is determined by the diameter of the laser used in the SPR instrument, i.e. 1 mm, we were able to calculate the area covered with BCG by multiplying the detection area with the percentage of the detection area covered with BCG (22.6% for the 6K-AH1 peptide, 13.6% for the 6K-TRP2 peptide and 11.0% for the CPP-SIINFEKL peptide).
8) Next, we calculated the footprint area of BCG based on its assumed diameter of 905.5 nm and obtained an area of approximately 643971 nm² per BCG particle.
9) By dividing the area covering the sensor area with BCG (obtained from point 7) with the footprint area of BCG (obtained from point 8) we obtained the number of BCG particles on the sensor surface. For the measurements for the different peptides in this study the corresponding number of BCG particles were: 275269 BCG particles (6K-AH1 peptide), 165397 BCG particles (6K-TRP2 peptide) and 133729 BCG particles (CPP-SIINFEKL peptide).
10) Hereafter, we calculated the number of peptides adsorbed to BCG from the SPR responses measured when 100 μM of the peptides was allowed to interact with the BCG layers. The SPR response values for the peptides could be converted to mass per area of adsorbed peptides by using a conversion factor of 600 ng/cm²×SPR response in degrees. The mass/area determined for the different peptides in this study were: 35.3 ng/cm² (6K-AH1 peptide), 301.6 ng/cm² (6K-TRP2 peptide) and 163.0 ng/cm² (CPP-SIINFEKL peptide).
11) By knowing the detection area, we could estimate the absolute mass of peptides adsorbed on BCG by multiplying the detection area with the mass/area of each peptide. The mass determined for the different peptides in this study were: approximately 0.277 ng (6K-AH1 peptide), 2.369 ng (6K-TRP2 peptide) and 1.280 ng (CPP-SIINFEKL peptide).
12) By knowing the molecular weight of the peptides (1868.21 g/mol for 6K-AH1 peptide, 1944.4 g/mol for 6K-TRP2 peptide, 3279.9 g/mol for CPP-SIINFEKL peptide) we were able to convert the mass to moles and finally to number of peptides by using the Avogadro constant. The number of peptides adsorbed for the different peptides in this study are: ca. 8.9×10¹⁰ (6K-AH1 peptide), 7.3×10¹¹ (6K-TRP2 peptide) and 2.4×10¹¹ (CPP-SIINFEKL peptide).
13) Finally, the number of peptides adsorbed per BCG particle was estimated by dividing the number of peptides (obtained from point 12) with the number of BCG particles obtained (from point 9).

Cross-Presentation and DC Activation Experiments:

For PeptiBAC cross-presentation experiments, 20 nmol of GRKKRRQRRRPQRWEKISIINFEKL [SEQ ID NO: 49] was complexed with BCG preparation for 15 min at 37° C., followed by removal of unbound peptides by centrifugation at 1000×G for 15 min to pellet the bacteria and removing the supernatant containing the unbound peptides. 10⁶ Jaws II cells were plated in 2 mL of complete alpha minimum essential media and were infected with the purified PeptiBAC preparation. After o/n incubation, cells were detached, washed and stained with either APC-conjugated anti-mouse H-2Kb bound to SIINFEKL [SEQ ID NO: 14] (141606, BioLegend), APC-conjugated mouse IgG k isotype Ctrl (400119, BioLegend), APC-conjugated anti-mouse CD40 antibody (17-0401-81, Ebioscience) or PerCpCy5.5-conjugated anti-mouse CD86 antibody (105028, Biolegend), PerCP-conjugated anti-mouse CD86 (105025, BioLegend) and FITC-conjugated anti-mouse CD40 (124607, BioLegend) antibodies and the stained samples were analyzed by flow cytometry.

ELISPOT Assays:

The amount of peptide-specific, e.g. SIINFEKL-specific, activated, interferon-gamma secreting T cells were measured by ELISPOT assay (CTL, USA) according to the manufacturer's instructions. Briefly, 2 ug of SIINFEKL peptide (SEQ ID NO: 14) was used to stimulate the antigen-presenting cells (NB. these peptides contained only the MHC class I epitope in order to be able to rule out any unspecific stimulation which could derive from CPP Tat sequence or immunoproteasome processing sequence used in the PeptiBAC platform). After 3 days of stimulation, plates where stained and sent to CTL-Europe GmbH for counting of the spots.

The amount of SIINFEKL ((SEQ ID NO: 14) OVA257-264), SVYDFFVWL ((SEQ ID NO:15) TRP2₁₈₀₋₁₈₈), BCG and adenovirus-specific activated, interferon-γ secreting T cells were measured by ELISPOT assay (CTL, Ohio USA) according to the manufacturer's instructions. Briefly, 2 μg of SIINFEKL or SVYDFFVWL peptide was used to stimulate the antigen presenting cells. After 2 or 3 days of stimulation, plates where stained and sent to CTL-Europe GmbH for counting of the spots.

The amount of peptide-specific, activated, interferon-gamma secreting T cells were measured by ELISPOT assay (ImmunoSpot, Bonn Germany) according to the manufacturer's instructions. Briefly, 2.5×10⁵ PBMCs were stimulated with a selected peptide (2 ug of each peptide) covering a conserving region in coronaviruses and tested in duplicate at 37° C. for 72 h. The spots were counted using an ELISpot reader system (ImmunoSpot, Bonn Germany) and background (DMSO only signal) corrected. The T cell responses are depicted as peptides specific reaction per 1×10⁶ PBMCs.

Flow Cytometry

The following antibodies were used in the experiments: TruStain FcX™ anti-mouse CD16/32 (101320, BioLegend), FITC anti-mouse CD8 (A502-3B-E, Proimmune), Phycoerythrin (PE) anti-mouse CD3e (550353, BD Pharmingen), Peridinin-Chlorophyll-Protein (PerCP) anti-mouse CD19 (115531, BioLegend) and PE-Cyanine 7 anti-mouse CD4 (25-0041-82 eBioscience). SIINFEKL epitope-specific T cells were studied using APC-labelled H-2Kb/SIINFEKL pentamer (F093-84B-E, Proimmune), SVYDFFVWL (Trp2) epitope-specific T cells were studied using PE-labelled H-2Kb/SVYDFFVWL pentamer (F185-82B-E, Proimmune), and SPSYVYHQF (a modified sequence derived from tumour rejection antigen AH1) epitope-specific T cells were studied using PE-labelled H-2Ld/SPSYVYHQF pentamer (F398-82A-E, Proimmune). Flow cytometric analysis were performed using a BD Accuri 6C Plus (BD Biosciences) or a BD LSRFortessa™ (BD Biosciences) flow cytometer and FlowJo software v10 (BD Biosciences) was used for the data analysis.

Bacterial Viability and Macrophage Assays

For the assessment of viability of the bacteria, CPP-containing peptide or poly-lysine-containing peptide was complexed with BCG (as described in the PeptiBAC complex formation-section) and complexes were directly plated for colony formation. Bacterial colonies were counted after 4 weeks of incubation at 37° C.

Mouse RAW-Blue macrophage reporter cell line (InvivoGen) expressing multiple pattern-recognition receptors (PRRs), including toll-like receptors (TLRs), NOD-like receptors (NLRs), RIG-I-like receptors (RLRs) and C-type lectin receptors (CLRs) was used to assess the activation NF-kB and AP-1 pathways induced by BCG and PeptiBAC. The presence of agonists of PRRs expressed by the RAW-Blue cells induce the activation of NF-kB and AP-1 leading to the secretion of embryonic alkaline phosphatase enzyme (SEAP). The substrate in the Quanti-BLUE (InvivoGen) system turns purple/blue in the presence of SEAP. The concentration of SEAP was measured using a multi-well plate reader (Varioskan Flash; ThermoLabsystems) to determine the relative activation efficacy of BCG and PeptiBAC. For the generation of bone-marrow derived macrophages (BMDMs), 10⁷ bone marrow cells isolated from C57BL/6JOlaHsd mouse were seeded in 10 ml of complete medium (RPMI-1640) (Sigma) containing 10 ng/mL recombinant macrophage colony-stimulating factor (Thermo Scientific), 10% FBS (Life Technologies), 2 mM L-glutamine, 50 U/mL penicillin, and 50 μg/mL streptomycin (Life Technologies). Cells were cultured at 37° C. in a humidified atmosphere of 5% CO₂. On day 3, half of the medium was replaced with fresh media. On day 6, part of the macrophages were harvested and used for cross-presentation experiments. For the rest of the macrophages, the media was gently aspirated and replaced with 10 mL of fresh complete medium containing 20 ng/ml interleukin-4 (IL-4, Life Technologies). Following 48 h of culture, M2 polarized macrophages were harvested and used for polarization experiments.

Animal Experiments

All animal experiments were reviewed and approved by the Experimental Animal Committee of the University of Helsinki and the Provincial Government of Southern Finland. C57BL/6JOlaHsd-mouse strain was used in all animal experiment. In B16.OVA animal experiments, 350000 B16.OVA-cells were injected in the right flank of mice and when the tumor size reached approximately 50 mm³ (10-12 days after injection) mice were treated with BCG, PeptiBAC-platform, peptides only or injection media only (Mock), specifically with 0.75-3×105 C.F.U/dose of BCG alone, 0.75-3×105 C.F.U/dose of PeptiBAC-OVA, peptides alone or PBS as a mock-treated group. Mice were treated on day 0, 2 and then a booster treatment was given on day 9. Tumors were measured every second day until the end of the experiment. In B16.F10 animal experiments, 150 000 B16.F10-cells were injected in the right flank of mice and when the tumor size reached approximately 50 mm³ (8-10 days after injection) mice were treated with BCG, various PeptiBAC-platforms or injection media only (Mock). Mice were treated on day 0, 3 and then a booster treatment was given on day 9. Tumors were measured every second day until the end of the experiment. On day 27 post tumour implantation, 3 mice from each group were sacrificed and spleens and tumours were collected for ELISPOT and flow cytometry analysis. The remaining animals were followed up for survival.

In B16.F10.K1 animal experiments, 300 000 B16.F10.K1-cells were injected in the right flank of mice together with a 1:1 ratio of Matrigel Basement Membrane Matrix High Concentration (Corning, USA), and when the tumor size reached approximately 50 mm³ (10-12 days after injection) mice were treated with BCG, BCG+immune checkpoint inhibitor (anti-PD-1), PeptiBAC, PeptiBAC+immune checkpoint inhibitor, immune checkpoint inhibitor alone or injection media only (Mock), specifically with 6.25×10⁶-12×10⁷ C.F.U/dose of BCG, 6.25×10⁶-12×10⁷ C.F.U/dose of PeptiBAC-Trp2 or PBS as a mock-treated group. Groups receiving anti-PD-1 (InVivoMab, USA, clone RMP1-14) were injected intraperitoneally three times per week with 100 μg/dose starting at day 16 post tumour implantation. Mice were treated on day 0, 2 and then a booster treatment was given on day 14. Immune checkpoint inhibitor was given intraperitoneally 3 times per week starting at day 5. Tumors were measured every second day until the end of the experiment.

For the CT26 colon experiment, 8- to 9-week-old immuno-competent female BALB/c mice were injected in the right flank with 600,000 CT26 cells, and were treated 11-, 13-, and 25-days post tumour implantation with 6.25×10⁶-12×10⁷ C.F.U/dose of BCG, 6.25×10⁶-12×10⁷ C.F.U/dose of PeptiBAC-AH1 or PBS as a mock-treated group. Groups receiving anti-PD-1 (InVivoMab, USA, clone RMP1-14) were injected intraperitoneally three times per week with 100 μg/dose starting at day 17 post tumour implantation. For the prime-boost vaccination experiments, 8- to 9-week-old immuno-competent naïve female C57BL/6JOlaHsd mice were treated subcutaneously with 1×10⁹ VP/dose of PeptiCRAd VALO-mD901-Trp2, PeptiCRAd VALO-mD901-OVA, 2-8×10⁶C.F.U/dose of PeptiBAC-Trp2, 2-8×10⁶C.F.U/dose of PeptiBAC-OVA or saline as a mock-treated group. Vaccinations were performed 14 days apart and 4 days after the last injection, mice were sacrificed, and spleens were collected for ELISPOT assay. All mice strains were obtained from Envigo (Venray, the Netherlands).

Results and Discussion:

Bacillus Calmette-Guérin Vaccine Prepared from an Attenuated Strain of Mycobacterium bovis can be Coated with Therapeutic Peptides by Using Cell-Penetrating Peptide Sequence or Polylysine or Polyarginine Linker Sequence as a Bacterial Membrane Attaching Anchor

As the outer membrane of mycobacteria consist of lipid-containing bilayer and the surface charge of the membrane is highly negative, we hypothesized that immunomodulatory/therapeutic peptides could be attached into the outer membrane by using either cell-penetrating peptide sequence (CPP) or highly positive amino acid sequence such as polylysine or a polyarginine stretch of six residues (6K) (see the schematic presentation of the attachment strategies used in FIGS. 1A-1C). In order to test this, we analyzed the anchoring affinities by surface plasmon resonance (SPR). SPR analysis showed high and stabile affinities towards the bacterial outer layer with both anchor moieties, with binding being completely dependent on the CPP or 6K moieties as peptide without either moieties did not interact with the bacterial outer layer (FIGS. 1D-1E).

Various CPP sequences were tested by surface plasmon resonance (SPR) for their efficacy to anchor therapeutic peptides into the mycobacterial cell wall (data not shown), and a CPP sequence derived from HIV Tat protein was found to be the most efficient CPP sequence for anchoring the peptides (FIGS. 2A-2C). In addition to CPP sequence derived from HIV Tat, positively charged polylysine sequence was found to efficiently anchor the peptides into the cell wall (FIGS. 2B and 2C). We also estimated the number of peptides bound to BCG bacterium using these two different attachment moieties and for the SIINFEKL antigen containing N-terminal CPP Tat sequence, the number of peptides bound to BCG was estimated to be 1.8×10⁶ peptide molecules/bacterium, and for the Trp2 antigen and for the AH1 antigen containing N-terminal polylysine sequences the number of peptides bound to BCG was estimated to be 4.4×10⁶ peptide molecules/bacterium and 3.2×10⁵ peptide molecules/bacterium, respectively.

In FIG. 2D various CPP sequences were tested by surface plasmon resonance (SPR) for their efficacy at anchoring therapeutic peptides into the mycobacterial cell wall, and a CPP sequence derived from HIV Tat protein was found to be the most efficient CPP sequence for anchoring the peptides. In addition to the CPP sequence derived from HIV Tat, a positively charged poly-lysine sequence was found to efficiently anchor the peptides into the cell wall. We estimated the number of peptides bound to BCG bacterium using these two different attachment moieties. For the SIINFEKL antigen containing an N-terminal CPP Tat sequence, the number of peptides bound to BCG was estimated to be 1.8×10⁶ peptide molecules/bacterium. For the Trp2 antigen and for the AH1 antigen containing N-terminal poly-lysine sequences, the number of peptides bound to BCG was estimated to be 4.4×10⁶ peptide molecules/bacterium and 3.2×10⁵ peptide molecules/bacterium, respectively.

Antigen-Presenting Cells can Efficiently Present Immunomodulatory Peptides Delivered by PeptiBAC

Next, we tested whether the modified BCG of the invention, PeptiBAC, can deliver immune-modulatory peptides into antigen-presenting cells (APC) and whether these peptides can readily be processed and cross-presented on the major histocompatibility complex I (MHC-I) molecules on the surface of the antigen-presenting cells. CPP-containing immunomodulatory peptide GRKKRRQRRRPQRWEKISIINFEKL [SEQ ID NO: 49] (which contains the neo-epitope SIINFEKL) was used to coat BCG to obtain PeptiBAC-OVA. PeptiBAC-OVA was then used to infect JAWS II dendritic cell (DC) line and the cross-presentation efficacy of the neo-epitope SIINFEKL was assessed by flow cytometry (FIG. 3A). SIINFEKL was efficiently cross-presented from PeptiBAC coated with CPP-conjugated SIINFEKL peptide (SEQ ID NO: 49), as approximately 40% of APCs were shown to cross-present the SIINFEKL epitope. In addition, PeptiBAC was shown to enhance the activation/maturation of the DCs compared to BCG as measured by the increased expression of the activation/maturation markers CD40 (FIG. 3B) and CD86 (FIG. 3C).

PeptiBAC Elicits Anti-Tumour Effects and Induces Robust Induction of Tumour-Specific CD8+ Effector T Cells in a Syngeneic Mouse Model of B16.OVA Melanoma

To study the anti-tumour efficacy of PeptiBAC platform, we used a well-established syngeneic mouse melanoma model B16 expressing chicken OVA as a model antigen. B16.OVA-tumour-bearing mice were treated intratumorally with OVA-targeted PeptiBAC (PeptiBAC-OVA), BCG, CPP-conjugated SIINFEKL peptides only or vehicle media (mock) (FIG. 4A). PeptiBAC-OVA-treated animals showed enhanced reduction in tumour growth as compared to all other treatment groups (FIGS. 4B-4E). In peptide only- and mock-treated groups, there was one responder in each group accounting for 12.5% response rate. In BCG-treated group the response rate was 25% and in the PeptiBAC-OVA-treated group the response rate was 37.5% (FIG. 4). Unexpectedly, BCG-treated mice had the lowest survival rate of all groups. In striking contrast, the survival rate of the PeptiBAC-OVA-treated mice was significantly enhanced as compared to other groups (FIG. 4F).

To further validate the PeptiBAC platform, systemic peptide-specific T cell response elicited by the different treatment groups was assessed using enzyme-linked immune absorbent spot (ELISpot) assay (FIGS. 5A-5B). Remarkably, PeptiBAC-OVA-treatment was able to induce massive systemic peptide-specific T cell response as measured by the number of SIINFEKL responsive T cells secreting interferon gamma (INF-G). Other treatments did not induce SIINFEKL-specific T cell response. In addition, the number of tumour infiltrating CD8⁺ T cells were assessed by flow cytometry and as compared to other groups, PeptiBAC-OVA-treated tumours showed increased T cell infiltration into the tumour microenvironment (TME).

Trivalent PeptiBAC Targeting Tumour Neoantigens and Helper T Cells Show Anti-Tumour Efficacy in Highly Immunosuppressive and Aggressive Mouse Model of B16.F10 Melanoma

In order to validate the PeptiBAC platform using tumour associated antigens such as ones derived from tyrosinase-related protein-2 (Trp2) and glycoprotein 100 (gp100) endogenously expressed by the B16.F10 melanoma, mice were engrafted with B16.F10 tumours and treated intratumorally with bivalent PeptiBAC targeting Trp2 and gp100 (PeptiBAC-TG), monovalent PeptiBAC targeting pan MHC class II molecules (PeptiBAC-P), trivalent PeptiBAC targeting Trp2, gp100 and pan MHC class II molecules (PeptiBAC-TGP), BCG and vehicle alone (mock) (FIG. 6A). PeptiBAC-TGP-treated animals showed enhanced reduction in tumour growth as compared to all other treatment groups. In BCG- and mock-treated groups, there was one responder in each group accounting for 12.5% response rate. In PeptiBAC-P treated group, no responders were seen. In PeptiBAC-TG-treated group the response rate was 25% and in the PeptiBAC-TGP-treated group the response rate was 50% (FIG. 6B). Similarly to the previous experiment with B16.OVA tumours, BCG-treated group showed the lowest survival rate as only 3/8 mice survived to the end of the experiment (day 21).

CPP-Containing but not Poly-Lysine-Containing Antigenic Peptides Reduce the Viability of BCG

The unexpected minimal efficacy seen using PeptiBAC with CPP-containing OVA antigen prompted us to test whether the CPP-containing antigen peptide could be toxic to the bacteria. Indeed, we saw a decrease in BCG viability when coated with CPP-containing antigen peptide but not when coated with poly-lysine-containing antigen peptide (FIGS. 7A & 7B). Poly-lysine extended SIINFEKL did not inhibit plaque formation, it was comparable to the control groups at both 20 μl and 100 μl (FIG. 7A). However, when poly-lysine extension is compared with the alternative use of CPP (FIG. 7B) it can be seen that use of the CPP linker drastically affects BCG viability (FIG. 7B).

To test whether this affect on viability was BCG specific we exposed E. coli (a Gram negative bacteria) and L. monocytogenes (a Gram positive bacteria) to CPP and measured viability, again using plaque or colony count and found CPP had no effect on E. coli or L. monocytogenes viability (FIG. 7C); colony count was commensurate with controls, indeed, slightly better than controls for E. coli (FIG. 7C), suggesting the inhibitory effect of CPP on BCG was specific for that bacteria.

To further validate the poly-lysine as a suitable attachment moiety, we tested the macrophage activation potential of PeptiBAC coated with poly-lysine containing antigen peptide. PeptiBAC (with poly-lysine-containing antigen peptide) was equally potent in activating NF-kB/AP1 pathways in murine RAW-blue macrophages as the non-coated BCG (FIG. 7B). As the tumour-associated macrophages (TAMs) are an important cell component of the TME, we also wanted to assess the cross-presentation properties of macrophages on PeptiBAC-delivered tumour antigens. PeptiBAC-OVA (BCG coated with poly-lysine-containing OVA peptide) was used to infect bone marrow-derived macrophages (BMDMs) for 24 h followed by the assessment of the cross-presentation efficacy of the epitope (SIINFEKL) by flow cytometry. Remarkably, PeptiBAC-delivered SIINFEKL was efficiently cross-presented on the surface of the BMDMs (FIG. 8A). In addition to macrophage presentation, we wanted to see whether PeptiBAC had the same properties as BCG on macrophage polarization from M2 state more towards the M1 state. M2 polarized macrophages were infected with BCG or PeptiBAC and the expression of macrophage M2 and M1 markers were analysed by flow cytometry. Both BCG and PeptiBAC were equally effective at polarizing M2 macrophages more towards the M1 state as assessed by the significant upregulation of both MHC-II and CD86 expression and by the significant downregulation of the M2 marker CD206 expression (FIG. 8B). Based on these data, poly-lysine was chosen as the attachment moiety to be used in all further experiments.

PeptiBAC Enhances Response Rate to Checkpoint Inhibitor Therapy in Therapy Resistant Mouse Model of B16.F10.K1 Melanoma

Finally, we tested the synergistic effect of PeptiBAC in combination with checkpoint inhibitor therapy using a mouse model of melanoma inherently resistant to checkpoint inhibitor therapy. B16.F10.K1-bearing mice were treated with Trp2-targeting PeptiBAC (PeptiBAC-Trp2, here, the attachment moiety was 6K sequence), PeptiBAC-Trp2 in combination with an anti-PD-1 antibody (checkpoint inhibitor against the PD-1/L-1 axis), BCG, BCG in combination with the anti-PD-1 antibody, the anti-PD-1 antibody alone or vehicle (mock). Although PeptiBAC-Trp2 treatment alone increased the number of responders to the treatment (44% response rate), the synergistic effect of PeptiBAC-Trp2 and the anti-PD-1 antibody increased the response rate even further (71% response rate). In all other groups, the response rate was 20% or below, including anti-PD-1 antibody alone (20% response rate) indicating efficient enhancement of checkpoint inhibitor therapy using PeptiBAC to relieve the therapy resistance (FIG. 10A, for individual tumour growth curves).

To evaluate the mechanism of tumour growth control, we assessed whether there was any differences in the Trp2-specific T cell responses between the treatment groups. We saw increased numbers of tumour-infiltrating CD4⁺ and CD8⁺ T cells in PeptiBAC-Trp2-treated tumours compared to BCG, anti-PD-1 alone and BCG in combination with anti-PD-1 ICI-treated tumours (FIG. 10A, upper panel). Also, the number of Trp2-specific CD8⁺ T cells was increased in PeptiBAC-Trp2-treated tumours compared to BCG, anti-PD-1 alone and BCG in combination with anti-PD-1 ICI-treated tumours (FIG. 10A, upper panel). In contrast to other treatment groups, PeptiBAC-Trp2 in combination with anti-PD-1-treated tumours had significantly more tumour-infiltrating CD4⁺ and CD8⁺ T cells as well as Trp2-specific CD8⁺ T cells, indicating a synergistic effect on T cell responses by combining the two treatment modalities (FIG. 10A, upper panel). We also evaluated systemic tumour-specific T cell responses by analysing the spleens of treated mice. No significant differences in the number of CD4⁺ and CD8⁺ T cells was found between groups. The number of Trp2-specific CD8⁺ T cells was increased in PeptiBAC-Trp2 in combination with anti-PD-1 ICI-treated spleens as compared to other treatment groups, again indicating a synergistic effect on T cell responses by combining the two treatment modalities (FIG. 10A, lower panel).

Intratumoural Treatment of PeptiBAC with Polylysine-Containing Modified Gp70 Antigen Increases the Number of Responders to Anti-PD-1 Therapy, Improves Tumour Control and Induces Tumour-Specific T Cell Responses in a Syngeneic Mouse Model of CT26 Colorectal Cancer

To validate the PeptiBAC platform as a more universal cancer vaccine platform, we tested the PeptiBAC platform in a syngeneic mouse model of CT26 colorectal cancer using a modified tumour rejection antigen AH1 in combination with anti-PD-1 immune checkpoint inhibitor therapy. AH1 represents one of the best characterized tumour rejection antigens in mice, and it is derived from the gp70 envelope protein of murine leukaemia virus (MuLV), which is endogenous in the genome of most laboratory mouse strains, including BALB/c strain used in these studies. Starting at 11 days post tumour engraftment, mice were treated intratumorally with BCG, anti-PD-1 alone, PeptiBAC-AH1, BCG in combination with anti-PD-1, PeptiBAC-AH1 in combination with anti-PD-1 or saline as a mock-treated group. Once again, the tumour size threshold was set to 450 mm³ for defining the responders in each treatment group. Mock, BCG, anti-PD-1 alone and BCG in combination with anti-PD-1 ICI-treated groups showed similar tumour growth characteristics with response rates of 25%, 22%, 25% and 10%, respectively. Interestingly, in contrast to the B16.F10.9/K1 melanoma model, PeptiBAC-AH1 treatment alone did not increase tumour growth control with response rate of 25%. Strikingly, PeptiBAC-AH1 in combination with anti-PD-1-treated animals showed very efficient tumour growth control with 80% response rate increasing the number of responders for anti-PD-1 therapy from 25% to 80% (FIG. 11A and FIG. 9 for average tumour growth curves). Again, we assessed whether there were any differences in T cell responses between the treatment groups. We saw no significant differences in the numbers of tumour infiltrating CD4+ and CD8+ T cells between the treatment groups although, interestingly, the number of CD8+ T cells in the PeptiBAC-AH1 treated tumours was slightly decreased as compared to tumours from other treatment groups. While the number of AH1-specific CD8+ T cells was slightly decreased in BCG and BCG in combination with anti-PD-1 ICI-treated tumours when compared to other treatment groups, PeptiBAC-AH1 in combination with anti-PD-1 ICI-treated tumours had significantly increased number of AH1-specific CD8+ T cells, suggesting a correlation between tumour growth control and number of AH1-specific CD8+ T cells in the TME (FIG. 11BI). Analysis of systemic tumour-specific T cell responses from the spleens of the treated mice showed no significant differences in the number of CD4+ and CD8+ T cells between groups. However, a significant increase of AH1-specific CD8+ T cells was seen in PeptiBAC-AH1 and PeptiBAC-AH1 in combination with anti-PD-1 ICI-treated mice spleens as compared to spleens from other groups (FIG. 11C).

Heterologous Prime-Boost Vaccination Strategy Combining PeptiBAC Platform with PeptiCRAd Platform Improves T Cell Responses Against the Coated Antigen

Finally, the PeptiBAC-platform was tested in combination with our recently described cancer vaccine platform PeptiCRAd (peptide-coated conditionally replicating adenovirus) using heterologous prime-boost vaccination strategy. By combining two immunologically distinct platforms coated with the same antigen, we tested whether this heterologous prime-boost approach could enhance T cell-specific immune responses in naïve mice towards the MHC-I restricted epitope presented by both platforms. To this end, we vaccinated naïve C57BL/6JOlaHsd mice with two doses of PeptiBAC-Trp2 or PeptiCRAd-Trp2 as homologous prime-boost controls or with PeptiBAC-Trp2 prime followed by PeptiCRAd-Trp2 boost and PeptiCRAd-Trp2 prime followed by PeptiBAC-Trp2 boost with doses given 14 days apart. 4 days after the boost dose, mice where sacrificed and the spleens were harvested and analysed for the induction of Trp2-specific T cell responses by the interferon-gamma ELISPOT. Vaccination with PeptiCRAd-Trp2 homologous prime-boost or PeptiCRAd-Trp2-PeptiBAC-Trp2 heterologous prime-boost did not induce significant Trp2-specific T cell responses in this vaccination setting. PeptiBAC-Trp2 homologous prime-boost vaccination induced moderate Trp2-specific T cell responses which were markedly enhanced by the PeptiBAC-Trp2-PeptiCRAd-Trp2 heterologous prime-boost vaccination regimen (FIG. 12A). Subsequently, we tested the same approach using the immunodominant epitope of ovalbumin (SIINFEKL), an epitope more immunogenic than Trp2, and assessed the induction of OVA-specific T cell responses again by using the interferon-gamma ELISPOT. Again, the heterologous prime-boost regimen induced significant enhancement of OVA-specific T cell responses compared to PeptiBAC-OVA vaccination (FIG. 12B).

Summary

BCG can be coated with therapeutic peptides (PeptiBAC) using a polylysine or polyarginine linker by attaching or anchoring said peptides in the bacterial membrane. Once administered to man this modified BCG is physiologically processed whereby Antigen-presenting cells can efficiently present immunomodulatory peptides delivered by PeptiBAC.

PeptiBAC with poly-lysine containing peptide antigen elicits anti-tumour effects and induces robust induction of tumour-specific CD8⁺ effector T cells in a melanoma and colon cancer mouse model.

Trivalent PeptiBAC with CPP-containing peptide antigens targeting tumour neoantigens and helper T cells show anti-tumour efficacy in a highly immunosuppressive and aggressive mouse model of melanoma.

Moreover, PeptiBAC with poly-lysine containing peptide antigen enhances the response rate to checkpoint inhibitor therapy in a known therapy resistant mouse model of melanoma.

Remarkably, PeptiBAC-Trp2 (poly-lysine containing Trp2 epitope peptide) treatment efficiently sensitized tumours to immune checkpoint inhibitor (ICI) therapy and this combination therapy group showed a response rate of 70%. In addition to increased tumour growth control, immunological analysis of the treated tumours revealed significant infiltration of CD4+, CD8+ as well as Trp2-specific CD8+ T cells into the TME of the PeptiBAC-Trp2+ICI-treated mice.

To further evaluate the PeptiBAC platform, we tested the platform in a syngeneic mouse model of CT26 colorectal cancer using a modified tumour rejection antigen AH1 (poly-lysine containing AH1 epitope peptide) in combination with anti-PD-1 ICI therapy. In this model, although we did not see effects on tumour growth control with either monotherapies, the combination of PeptiBAC-AH1 and anti-PD-1 ICI had a remarkable synergistic effect showing a response rate of 80%. In addition, the combination-treated mice showed significantly increased infiltration of AH1-specific CD8+ T cells into the TME. Both PeptiBAC-AH1 monotherapy and PeptiBAC-AH1 in combination with anti-PD-1 significantly increased AH1-specific CD8+ T cells in spleens as compared to other treatment groups.

Heterologous prime-boost vaccination, sequentially using two or more immunologically distinct platforms to deliver the antigen(s) was undertaken and showed the PeptiBAC (with poly-lysine containing antigen peptide) platform could be used as a component of a heterologous prime-boost vaccination setting together with another peptide-based cancer vaccine platform e.g. using oncolytic adenoviruses (called PeptiCRAd). Interestingly, we saw enhanced antigen-specific T cell responses when compared to a homologous prime-boost vaccination with PeptiBAC only, when PeptiBAC was used as a priming vaccine and PeptiCRAd as a booster vaccine.

Taken together, these results teach that PeptiBAC is superior at triggering anti-disease effects particularly anti-tumour effects in instances where the disease is particularly aggressive such as in a highly immunosuppressive and aggressive disease or where a disease is known to be resistant to therapy. Further, PeptiBAC is particularly effective when combined with an immune checkpoint inhibitor therapy or when used in a heterologous prime-boost vaccination regimen.

TABLE 1
List of SARS-CoV2 peptides.

| | | | The length |
|---|---|---|---|
| | | | of Poly- |
| | | | lysine tail |
| Source | | Full amino acid | used in |
| protein | Short name | sequence | experiments |
| AP3A | AP3A_GLEAP | GLEAPFLYLYALVYFLQS | 9K |
| | | INFV | |
| R1AB | R1AB_GLVAE | GLVAEWFLAYILFTRFFY | 9K |
| | | VL | |
| VME1 | VME1_LPKEI | LPKEITVATSRTLSYYKL | 6K |
| | | GA | |
| NCAP | NCAP_AQFAP | AQFAPSASAFFGMSRIGM | 6K |
| | | EV | |
| NCAP | NCAP_VILLN | VILLNKHIDAYKTFPPTE | 6K |
| | | PK | |
| R1AB | R1AB_KVTLV | KVTLVFLFVAAIFYLITP | 6K |
| | | VHVMSK | |
| R1A | R1A_KRAKV | KRAKVTSAMQTMLFTMLR | 6K |
| | | KL | |
| NCAP | NCAP_ALALL | ALALLLLDRLNQLESKMS | 6K |
| | | GK | |
| VME1 | VME1_KLIFL | KLIFLWLLWPVTLACFVL | 9K |
| | | AAV | |

TABLE 2
List of priority for peptides (based on SPR
binding data presented in FIGS. 13A-13B, high
binders are listed first):

```
  1. R1AB_GLVAE
  2. AP3A_GLEAP
  3. R1AB_KVTLV
  4. VME1_KLIFL
5. R1A_KRAKV
  6. VME1_LPKEI
  7. NCAP_AQFAP
  8. NCAP_VILLN
  9. NCAP_ALALL
```

1. An attenuated Mycobacterium bovis of the strain Bacillus Calmette-Guérin (BCG) for use in humans to prevent or treat a disease wherein said BCG is coated with a plurality of peptide antigens capable of eliciting an immune reaction against said disease in said human and wherein said plurality of peptide antigens are attached to said bacteria using a poly-lysine or poly-arginine peptide linker.

2. The attenuated BCG according to claim 1, wherein said poly-lysine or poly-arginine linker comprises at least 4, 5, 6, 7, 8, or 9 lysines or arginines, respectively.

3. The attenuated BCG according to claim wherein said linker consists of 6 lysines or 6 arginines.

4. The attenuated BCG according to claim 1, wherein said attenuated BCG is coated with a plurality of different peptide antigens.

5. The attenuated BCG according to claim 1, wherein at least one of said plurality of peptide antigens is MHC-I or MHC-II restricted.

6. The attenuated BCG according to claim 1, wherein said disease is an infection.

7. The attenuated BCG according to claim 6, wherein said infection is a respiratory infection such as a corona virus infection (e.g. SARS-CoV-2).

8. The attenuated BCG according to claim wherein said disease is a viral infection and said plurality of peptide antigens is/are derived from at least one of the following proteins: VME1, AP3A, R1AB, NS7B, NCAP, R1A and viral Spike proteins.

9. The attenuated BCG according to claim 6, wherein said disease is an infection and said plurality of peptide antigens comprises at least one of the following peptides:
[SEQ ID NO: 7]
GLVAEWFLAYILFTRFFYVL derived from R1AB;
[SEQ ID NO: 4]
GLEAPFLYLYALVYFLQSINFV derived from AP3A;
[SEQ ID NO: 6]
KVTLVFLFVAAIFYLITPVHVMSK derived from R1AB;
[SEQ ID NO: 2]
KLIFLWLLWPVTLACFVLAAV derived from VME1;
[SEQ ID NO: 8]
KRAKVTSAMQTMLFTMLRKL derived from R1A;
[SEQ ID NO: 3]
LPKEITVATSRTLSYYKLGA derived from VME1;
[SEQ ID NO: 11]
AQFAPSASAFFGMSRIGMEV derived from NCAP;
[SEQ ID NO: 13]
VILLNKHIDAYKTFPPTEPK derived from NCAP_;
[SEQ ID NO: 12]
ALALLLLDRLNQLESKMSGK derived from NCAP;
[SEQ ID NO: 1]
IAMACLVGLMWLSYFIASFRLFAR derived from VME1;
[SEQ ID NO: 5]
QMAPISAMVRMYIFFASFYYVWK derived from R1AB;
[SEQ ID NO: 9]
EIPVAYRKVLLRKNGNKGAG derived from R1AB;
[SEQ ID NO: 10]
ELSLIDFYLCFLAFLLFLVLIMLII derived from NS7B; and
a polypeptide that is at least 60% identical
with one of the afore peptides.

10. The attenuated BCG according to claim wherein said last polypeptide has 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% identity with any one of the afore peptides.

11. The attenuated BCG according to claim 1, wherein said disease is cancer and said peptide antigens are selected from the group comprising tumour associated antigens (TAAs), tumour-specific antigens (TSAs) or neoantigens.

12. The attenuated BCG according to claim 11, wherein said plurality of peptide antigens comprises at least one of the following polypeptides:
i)
[SEQ ID NO: 14]
SIINFEKL;
ii)
[SEQ ID NO: 15]
SVYDFFVWL;
iii)
[SEQ ID NO: 16]
KVPRNQDWL;
iv)
[SEQ ID NO: 56]
SPSYVYHQF;
or
v)
a polypeptide that is at least 60% identical with
the peptides of parts i, ii, iii or iv.

13. The attenuated BCG according to claim 12, wherein said polypeptide of v) has 61, 62, 63, 64, 65, 66, 67, 68, 69 70, 71, 72, 73, 74, 75, 76, 77, 78, 79, 80, 81, 82, 83, 84, 85, 86, 87, 88, 89, 90, 91, 92 93, 94, 95, 96, 97, 98 or 99% identity with the peptides of i), ii) iii) or iv).

14. The attenuated BCG according to claim 1, wherein said plurality of peptide antigens comprises at least one peptide antigen comprising AKFVAAWTLKAAA (Padre PEPTIDE) [SEQ ID NO:17].

15. The attenuated BCG according to claim 11, wherein said cancer is any one or more of the following cancers: nasopharyngeal cancer, synovial cancer, hepatocellular cancer, renal cancer, cancer of connective tissues, melanoma, lung cancer, bowel cancer, colon cancer, rectal cancer, colorectal cancer, brain cancer, throat cancer, oral cancer, liver cancer, bone cancer, pancreatic cancer, choriocarcinoma, gastrinoma, pheochromocytoma, prolactinoma, T-cell leukemia/lymphoma, neuroma, von Hippel-Lindau disease, Zollinger-Ellison syndrome, adrenal cancer, anal cancer, bile duct cancer, bladder cancer, ureter cancer, oligodendroglioma, neuroblastoma, meningioma, spinal cord tumor, osteochondroma, chondrosarcoma, Ewing's sarcoma, cancer of unknown primary site, carcinoid, carcinoid of gastrointestinal tract, fibrosarcoma, breast cancer, Paget's disease, cervical cancer, esophagus cancer, gall bladder cancer, head cancer, eye cancer, neck cancer, kidney cancer, Wilms' tumor, liver cancer, Kaposi's sarcoma, prostate cancer, testicular cancer, Hodgkin's disease, non-Hodgkin's lymphoma, skin cancer, mesothelioma, multiple myeloma, ovarian cancer, endocrine pancreatic cancer, glucagonoma, parathyroid cancer, penis cancer, pituitary cancer, soft tissue sarcoma, retinoblastoma, small intestine cancer, stomach cancer, thymus cancer, thyroid cancer, trophoblastic cancer, hydatidiform mole, uterine cancer, endometrial cancer, vagina cancer, vulva cancer, acoustic neuroma, mycosis fungoides, insulinoma, carcinoid syndrome, somatostatinoma, gum cancer, heart cancer, lip cancer, meninges cancer, mouth cancer, nerve cancer, palate cancer, parotid gland cancer, peritoneum cancer, pharynx cancer, pleural cancer, salivary gland cancer, tongue cancer and tonsil cancer.

16. The attenuated BCG according to claim 1, wherein the number of peptides bound to each BCG is greater than 1.8×10⁶ peptide molecules/bacterium and ideally greater than 2×10⁶, 3×10⁶, or 4×10⁶ peptide molecules/bacterium.

17. A pharmaceutical composition comprising the attenuated BCG according to claim 1 and a suitable carrier.

18. The pharmaceutical composition according to claim 17 which is formulated for intradermal, intranasal, subcutaneous, percutaneous, intratumoral, intramuscular, intra-arterial, intravenous, intrapleural, intravesicular, intracavitary or peritoneal injection, or oral administration.

19. A method of treating a disease in an individual comprising, administering to the individual an effective amount of the attenuated BCG according to claim 1.

20. The method according to claim 19, further comprising administering to the individual a checkpoint modulator or an immune checkpoint inhibitor.

21. The method according to claim 20, wherein the administration of said attenuated BCG is preceded by and/or followed by the administration of the checkpoint modulator molecule or the immune checkpoint inhibitor; or said attenuated BCG is co-administered with the checkpoint modulator molecule or the immune checkpoint inhibitor.

22. A combination therapeutic comprising the attenuated BCG according to claim 1, and at least one checkpoint modulator or an immune checkpoint inhibitor.

23. The combination therapeutic according to claim 22 wherein said checkpoint modulator or the immune checkpoint inhibitor is cytotoxic T-lymphocyte protein 4 (CTLA-4) or programmed cell death protein 1 pathway (PD-1/PD-L1).

24. (canceled)

25. (canceled)

26. The method of claim 19, wherein the disease is a cancer, infection, respiratory disease, influenza, TB, influenza, common cold, or coronavirus infection comprising SARS and MERS.

27. A method for vaccinating a subject against a disease, comprising:
i) administering to said subject the attenuated BCG according to claim 1, wherein said BCG is coated with a plurality of peptide antigens capable of eliciting an immune reaction against said disease; and
ii) prior to step i) or after step i), administering to said subject the attenuated BCG according to claim 1, wherein said BCG is coated with a plurality of different peptide antigens, compared to the BCG or the pharmaceutical composition of part i), capable of eliciting an immune reaction against said disease; or
iii) prior to step i) or after step i), administering to said subject a viral vector wherein said vector is coated with a plurality of peptide antigens, compared to the BCG or the pharmaceutical composition of part i), capable of eliciting an immune reaction against said disease; or
iv) prior to step i) or after step i), administering to said subject a vaccine comprising at least one antigen capable of eliciting an immune response against said disease.

28. The method according to claim 27 wherein the vector of part iii) is coated with the same peptide antigens as the BCG or the pharmaceutical composition of part i); or the vector of part iii) is coated with different peptide antigens compared with peptide antigens coating the BCG or the pharmaceutical composition of part i), but capable of eliciting an immune response against said disease.

29. The method of claim 27, wherein said vector is an attenuated virus.


SEQUENCE LISTING (67 sequences)

SEQ ID NO: 1 - protein, 24 aa, artificial sequence
  Peptide sequence
IAMACLVGLMWLSYFIASFRLFAR

SEQ ID NO: 2 - protein, 21 aa, artificial sequence
  Peptide sequence
KLIFLWLLWPVTLACFVLAAV

SEQ ID NO: 3 - protein, 20 aa, artificial sequence
  Peptide sequence
LPKEITVATSRTLSYYKLGA

SEQ ID NO: 4 - protein, 22 aa, artificial sequence
  Peptide sequence
GLEAPFLYLYALVYFLQSINFV

SEQ ID NO: 5 - protein, 23 aa, artificial sequence
  Peptide sequence
QMAPISAMVRMYIFFASFYYVWK

SEQ ID NO: 6 - protein, 24 aa, artificial sequence
  Peptide sequence
KVTLVFLFVAAIFYLITPVHVMSK

SEQ ID NO: 7 - protein, 20 aa, artificial sequence
  Peptide sequence
GLVAEWFLAYILFTRFFYVL

SEQ ID NO: 8 - protein, 20 aa, artificial sequence
  Peptide sequence
KRAKVTSAMQTMLFTMLRKL

SEQ ID NO: 9 - protein, 20 aa, artificial sequence
  Peptide sequence
EIPVAYRKVLLRKNGNKGAG

SEQ ID NO: 10 - protein, 25 aa, artificial sequence
  Peptide sequence
ELSLIDFYLCFLAFLLFLVLIMLII

SEQ ID NO: 11 - protein, 20 aa, artificial sequence
  Peptide sequence
AQFAPSASAFFGMSRIGMEV

SEQ ID NO: 12 - protein, 20 aa, artificial sequence
  Peptide sequence
ALALLLLDRLNQLESKMSGK

SEQ ID NO: 13 - protein, 20 aa, artificial sequence
  Peptide sequence
VILLNKHIDAYKTFPPTEPK

SEQ ID NO: 14 - protein, 8 aa, artificial sequence
  Peptide sequence
SIINFEKL

SEQ ID NO: 15 - protein, 9 aa, artificial sequence
  Peptide sequence
SVYDFFVWL

SEQ ID NO: 16 - protein, 9 aa, artificial sequence
  Peptide sequence
KVPRNQDWL

SEQ ID NO: 17 - protein, 13 aa, artificial sequence
  Peptide sequence
AKFVAAWTLKAAA

SEQ ID NO: 18 - protein, 33 aa, artificial sequence
  Peptide sequence
KKKKKKKKKIAMACLVGLMWLSYFIASFRLFAR

SEQ ID NO: 19 - protein, 30 aa, artificial sequence
  Peptide sequence
KKKKKKKKKKLIFLWLLWPVTLACFVLAAV

SEQ ID NO: 20 - protein, 29 aa, artificial sequence
  Peptide sequence
KKKKKKKKKLPKEITVATSRTLSYYKLGA

SEQ ID NO: 21 - protein, 31 aa, artificial sequence
  Peptide sequence
KKKKKKKKKGLEAPFLYLYALVYFLQSINFV

SEQ ID NO: 22 - protein, 32 aa, artificial sequence
  Peptide sequence
KKKKKKKKKQMAPISAMVRMYIFFASFYYVWK

SEQ ID NO: 23 - protein, 33 aa, artificial sequence
  Peptide sequence
KKKKKKKKKKVTLVFLFVAAIFYLITPVHVMSK

SEQ ID NO: 24 - protein, 29 aa, artificial sequence
  Peptide sequence
KKKKKKKKKGLVAEWFLAYILFTRFFYVL

SEQ ID NO: 25 - protein, 29 aa, artificial sequence
  Peptide sequence
KKKKKKKKKKRAKVTSAMQTMLFTMLRKL

SEQ ID NO: 26 - protein, 29 aa, artificial sequence
  Peptide sequence
KKKKKKKKKEIPVAYRKVLLRKNGNKGAG

SEQ ID NO: 27 - protein, 34 aa, artificial sequence
  Peptide sequence
KKKKKKKKKELSLIDFYLCFLAFLLFLVLIMLII

SEQ ID NO: 28 - protein, 29 aa, artificial sequence
  Peptide sequence
KKKKKKKKKAQFAPSASAFFGMSRIGMEV

SEQ ID NO: 29 - protein, 29 aa, artificial sequence
  Peptide sequence
KKKKKKKKKALALLLLDRLNQLESKMSGK

SEQ ID NO: 30 - protein, 29 aa, artificial sequence
  Peptide sequence
KKKKKKKKKVILLNKHIDAYKTFPPTEPK

SEQ ID NO: 31 - protein, 17 aa, artificial sequence
  Peptide sequence
KKKKKKKKKSIINFEKL

SEQ ID NO: 32 - protein, 18 aa, artificial sequence
  Peptide sequence
KKKKKKKKKSVYDFFVWL

SEQ ID NO: 33 - protein, 18 aa, artificial sequence
  Peptide sequence
KKKKKKKKKKVPRNQDWL

SEQ ID NO: 34 - protein, 22 aa, artificial sequence
  Peptide sequence
KKKKKKKKKAKFVAAWTLKAAA

SEQ ID NO: 35 - protein, 20 aa, artificial sequence
  Peptide sequence
GLWRALWRLLRSLWRLLWRA

SEQ ID NO: 36 - protein, 16 aa, artificial sequence
  Peptide sequence
RQIKIWFQNRRMKWKK

SEQ ID NO: 37 - protein, 18 aa, artificial sequence
  Peptide sequence
KLALKLALKALKAALKLA

SEQ ID NO: 38 - protein, 12 aa, artificial sequence
  Peptide sequence
GRKKRRQRRRPQ

SEQ ID NO: 39 - protein, 19 aa, artificial sequence
  Peptide sequence
KKKKKKAKFVAAWTLKAAA

SEQ ID NO: 40 - protein, 26 aa, artificial sequence
  Peptide sequence
KKKKKKVILLNKHIDAYKTFPPTEPK

SEQ ID NO: 41 - protein, 15 aa, artificial sequence
  Peptide sequence
KKKKKKKVPRNQDWL

SEQ ID NO: 42 - protein, 14 aa, artificial sequence
  Peptide sequence
KKKKKKSIINFEKL

SEQ ID NO: 43 - protein, 15 aa, artificial sequence
  Peptide sequence
KKKKKKSVYDFFVWL

SEQ ID NO: 44 - protein, 15 aa, artificial sequence
  Peptide sequence
KKKKKKSPSYAYHQF

SEQ ID NO: 45 - protein, 9 aa, artificial sequence
  Peptide sequence
SPSYAYHQF

SEQ ID NO: 46 - protein, 27 aa, artificial sequence
  Peptide sequence
KKKKKKKLIFLWLLWPVTLACFVLAAV

SEQ ID NO: 47 - protein, 26 aa, artificial sequence
  Peptide sequence
KKKKKKLPKEITVATSRTLSYYKLGA

SEQ ID NO: 48 - protein, 30 aa, artificial sequence
  Peptide sequence
KKKKKKIAMACLVGLMWLSYFIASFRLFAR

SEQ ID NO: 49 - protein, 25 aa, artificial sequence
  Peptide sequence
GRKKRRQRRRPQRWEKISIINFEKL

SEQ ID NO: 50 - protein, 26 aa, artificial sequence
  Peptide sequence
GRKKRRQRRRPQRWEKISVYDFFVWL

SEQ ID NO: 51 - protein, 26 aa, artificial sequence
  Peptide sequence
GRKKRRQRRRPQRWEKIKVPRNQDWL

SEQ ID NO: 52 - protein, 27 aa, artificial sequence
  Peptide sequence
GRKKRRQRRRPQRRAKFVAAWTLKAAA

SEQ ID NO: 53 - protein, 34 aa, artificial sequence
  Peptide sequence
GRKKRRQRRRPQRRAKFVAAWTLKAAAKVPRNQD

SEQ ID NO: 54 - protein, 36 aa, artificial sequence
  Peptide sequence
GRKKRRQRRRPQRRAKFVAAWTLKAAASVYDFFVWL

SEQ ID NO: 55 - protein, 13 aa, artificial sequence
  Peptide sequence
RWEKISIINFEKL

SEQ ID NO: 56 - protein, 9 aa, artificial sequence
  Peptide sequence
SPSYVYHQF

SEQ ID NO: 57 - protein, 18 aa, artificial sequence
  Peptide sequence
KKKKKKKKKSPSYVYHQF

SEQ ID NO: 58 - protein, 15 aa, artificial sequence
  Peptide sequence
KKKKKKSPSYVYHQF

SEQ ID NO: 59 - protein, 26 aa, artificial sequence
  Peptide sequence
KKKKKKALALLLLDRLNQLESKMSGK

SEQ ID NO: 60 - protein, 28 aa, artificial sequence
  Peptide sequence
KKKKKKGLEAPFLYLYALVYFLQSINFV

SEQ ID NO: 61 - protein, 29 aa, artificial sequence
  Peptide sequence
KKKKKKQMAPISAMVRMYIFFASFYYVWK

SEQ ID NO: 62 - protein, 30 aa, artificial sequence
  Peptide sequence
KKKKKKKVTLVFLFVAAIFYLITPVHVMSK

SEQ ID NO: 63 - protein, 26 aa, artificial sequence
  Peptide sequence
KKKKKKGLVAEWFLAYILFTRFFYVL

SEQ ID NO: 64 - protein, 26 aa, artificial sequence
  Peptide sequence
KKKKKKKRAKVTSAMQTMLFTMLRKL

SEQ ID NO: 65 - protein, 26 aa, artificial sequence
  Peptide sequence
KKKKKKEIPVAYRKVLLRKNGNKGAG

SEQ ID NO: 66 - protein, 31 aa, artificial sequence
  Peptide sequence
KKKKKKELSLIDFYLCFLAFLLFLVLIMLII

SEQ ID NO: 67 - protein, 26 aa, artificial sequence
  Peptide sequence
KKKKKKAQFAPSASAFFGMSRIGMEV